SEQUENCE LISTING (56 sequences)

SEQ ID NO: 1 - DNA, 25 bp, synthetic construct
  source 1..25: mol_type = other DNA
aaaaaaaaaaaaaaaaaaaaaaaaa

SEQ ID NO: 2 - DNA, 21 bp, synthetic construct
  source 1..21: mol_type = other DNA
aaaaaaaaaaataaaaaaaaa

SEQ ID NO: 3 - DNA, 32 bp, synthetic construct
  source 1..32: mol_type = other DNA
tggggatcatccctatagtgagtcgtattagt

SEQ ID NO: 4 - DNA, 22 bp, synthetic construct
  source 1..22: mol_type = other DNA
aaaaaaaaaaaaaaaaaaaacg

SEQ ID NO: 5 - DNA, 26 bp, synthetic construct
  misc_feature 1..3: note = RNA
  source 1..26: mol_type = other DNA
gcgaaaaaaaaaaataaaaaaaaacg

SEQ ID NO: 6 - DNA, 29 bp, synthetic construct
  misc_feature 1..3: note = RNA
  source 1..29: mol_type = other DNA
gcgaaaaaaaaaaaaaaaaaaaaaaaaaa

SEQ ID NO: 7 - DNA, 26 bp, synthetic construct
  misc_feature 1..3: note = RNA
  source 1..26: mol_type = other DNA
gcgtaaaaaaaaaaaaaaaaaaaacg

SEQ ID NO: 8 - DNA, 25 bp, synthetic construct
  misc_feature 1..3: note = RNA
  source 1..25: mol_type = other DNA
gcgaaaaaaaaaaaaaaaaaaaacg

SEQ ID NO: 9 - RNA, 20 nt, synthetic construct
  source 1..20: mol_type = other RNA
aaaaaaaaaaaaaaaaaaaa

SEQ ID NO: 10 - DNA, 111 bp, synthetic construct
  misc_feature 1..87: note = RNA
  source 1..111: mol_type = other DNA
gcaagatggcggcagtgagcggctggcggctgttcaagaagattagcggcagcagcggcggtaactatcagctcgctttcttgctgaaaaaaaaaaaaaaaaaaaaaaaaa

SEQ ID NO: 11 - RNA, 111 nt, synthetic construct
  source 1..111: mol_type = other RNA
gcaagatggcggcagtgagcggctggcggctgttcaagaagattagcggcagcagcggcggtaactatcagctcgctttcttgctgaaaaaaaaaaaaaaaaaaaaaaaaa

SEQ ID NO: 12 - DNA, 65 bp, synthetic construct
  misc_feature 1..44: note = RNA
  source 1..65: mol_type = other DNA
gggagagccaccatggtgagcggctggtgttcaagaagattagcaaaaaaaaaaataaaaaaaaa

SEQ ID NO: 13 - RNA, 64 nt, synthetic construct
  source 1..64: mol_type = other RNA
gggagagccaccatggtgagcggctggtgttcaagattagctgaaaaaaaaaaaaaaaaaaaaa

SEQ ID NO: 14 - DNA, 32 bp, synthetic construct
  source 1..32: mol_type = other DNA
tggggatcatccctatagtgactcctattagt

SEQ ID NO: 15 - DNA, 20 bp, synthetic construct
  source 1..20: mol_type = other DNA
tcaggtggaggaagttctgg

SEQ ID NO: 16 - DNA, 105 bp, synthetic construct
  source 1..105: mol_type = other DNA
ggagagccaccatgtcaggaggaagttctggtgtgagcggctggcggctgttcaagaagattagcggttccagtggtggtggatctaaaaaaaaaaaaaaaaaaa

SEQ ID NO: 17 - DNA, 20 bp, synthetic construct
  source 1..20: mol_type = other DNA
caaggtcaccaccacctaga

SEQ ID NO: 18 - protein, 26 aa, synthetic construct
MSGGGSSGVSGWRLFKKISGSSGGGS

SEQ ID NO: 19 - protein, 7 aa, synthetic construct
SGGGSSG

SEQ ID NO: 20 - protein, 7 aa, synthetic construct
GSSGGGS

SEQ ID NO: 21 - RNA, 51 nt, synthetic construct
  source 1..51: mol_type = other RNA
gggagagccaccatggtgagcggctggcggctgttcaagaagattagctga

SEQ ID NO: 22 - RNA, 71 nt, synthetic construct
  source 1..71: mol_type = other RNA
gggagagccaccatggtgagcggctggcggctgttcaagaagattagctgaaaaaaaaaaaaaaaaaaaaa

SEQ ID NO: 23 - DNA, 69 bp, synthetic construct
  misc_feature 1..48: note = RNA
  source 1..69: mol_type = other DNA
gggagagccaccatggtgagcggctggcggctgttcaagaagattagcaaaaaaaaaaataaaaaaaaa

SEQ ID NO: 24 - DNA, 173 bp, synthetic construct
  source 1..173: mol_type = other DNA
ggtggatctactctgctatttttgcgggcttgtaaccgctttattgccgaagcaataaagctgcattagatccaccaccactggaaccgctaatcttcttgaacagccgccagccgctcacaccagaacttcctccacctgacatggtggctctccctatagtgagtcgtatt

SEQ ID NO: 25 - DNA, 32 bp, synthetic construct; identical to SEQ ID NO: 3
  source 1..32: mol_type = other DNA

SEQ ID NO: 26 - DNA, 130 bp, synthetic construct
  source 1..130: mol_type = other DNA
tttttttttttttttttttttcaagatccaccaccactggaaccgctaatcttcttgaacagccgccagccgctcacaccagaacttcctccacctgacatggtggctctccctatagtgagtcgtatta

SEQ ID NO: 27 - RNA, 113 nt, synthetic construct
  source 1..113: mol_type = other RNA
gggagagccaccatgtcaggtggaggaagttctggtgtgagcggctggcggctgttcaagaagattagcggttccagtggtggtggatcttgaaaaaaaaaaaaaaaaaaaaa

SEQ ID NO: 28 - RNA, 73 nt, synthetic construct
  source 1..73: mol_type = other RNA
gcaagatggcggcagtgagcggctggcggctgttcaagaagattagcggcagcagcggcggctaactatatca

SEQ ID NO: 29 - DNA, 30 bp, synthetic construct
  source 1..30: mol_type = other DNA
agcgagctgatatagttagccgccgctgct

SEQ ID NO: 30 - RNA, 98 nt, synthetic construct
  source 1..98: mol_type = other RNA
gcaagatggcggcagtgagcggctggcggctgttcaagaagattagcggcagcagcggcggctaactatatcaaaaaaaaaaaaaaaaaaaaaaaaaa

SEQ ID NO: 31 - RNA, 41 nt, synthetic construct
  source 1..41: mol_type = other RNA
gctcgctttcttgctgaaaaaaaaaaaaaaaaaaaaaaaaa

SEQ ID NO: 32 - DNA, 41 bp, synthetic construct
  misc_feature 1..16: note = RNA
  source 1..41: mol_type = other DNA
gctcgctttcttgctgaaaaaaaaaaaaaaaaaaaaaaaaa

SEQ ID NO: 33 - RNA, 41 nt, synthetic construct; identical to SEQ ID NO: 31
  source 1..41: mol_type = other RNA

SEQ ID NO: 34 - DNA, 41 bp, synthetic construct; identical to SEQ ID NO: 32
  misc_feature 1..16: note = RNA
  source 1..41: mol_type = other DNA

SEQ ID NO: 35 - RNA, 16 nt, synthetic construct
  source 1..16: mol_type = other RNA
gctcgctttcttgctg

SEQ ID NO: 36 - RNA, 20 nt, synthetic construct
  source 1..20: mol_type = other RNA
gctcgctttcttgctgaaaa

SEQ ID NO: 37 - RNA, 20 nt, synthetic construct
  source 1..20: mol_type = other RNA
gctcgctttcttgctgaaaa

SEQ ID NO: 38 - DNA, 20 bp, synthetic construct
  misc_feature 1..16: note = RNA
  source 1..20: mol_type = other DNA
gctcgctttcttgctgaaaa

SEQ ID NO: 39 - RNA, 41 nt, synthetic construct; identical to SEQ ID NO: 31
  source 1..41: mol_type = other RNA

SEQ ID NO: 40 - DNA, 41 bp, synthetic construct; identical to SEQ ID NO: 32
  misc_feature 1..41: note = RNA
  source 1..41: mol_type = other DNA

SEQ ID NO: 41 - DNA, 44 bp, synthetic construct
  misc_feature 1..16: note = RNA
  source 1..44: mol_type = other DNA
gctcgctttcttgctgaaaaaaaaaaaaaaaaaaaaaaaaaaaa

SEQ ID NO: 42 - DNA, 248 bp, synthetic construct
  source 1..248: mol_type = other DNA
taatacgactcactatagcaagatggcggcaatgaagtgggtgaccttcatcagcctgctgttcctgttcagcagcgcctacagccatggagagggcacctttacctctgatgtgtccagctacctggaaggccaggctgccaaagagtttatcgcctggctggtcaaaggcagaggctaaaatgcagctttattgcttcggcaataaagcggttacaagcccgcaaaaatagcagagtatagcctct

SEQ ID NO: 43 - DNA, 311 bp, synthetic construct
  source 1..311: mol_type = other DNA
atagtggattggccaggtccattttaatacgactcactatagcaagatggcggcaatgaagatcatcctgtggctgtgtgtgttcggcctgttcctggccaccctgttccccatcagctggcagaagcgcatcaagcgccatggagagggcacctttacctctgatgtgtccagctacctggaaggccaggctgccaaagagtttatcgcctggctggtcaaaggcagaggctaaaagattagcaatgcagctttattgcttcggcaataaagcggttacaagcccgcaaaaatagcagagtactaatctt

SEQ ID NO: 44 - DNA, 311 bp, synthetic construct; identical to SEQ ID NO: 43
  source 1..311: mol_type = other DNA

SEQ ID NO: 45 - DNA, 311 bp, synthetic construct; identical to SEQ ID NO: 43
  source 1..311: mol_type = other DNA

SEQ ID NO: 46 - DNA, 29 bp, synthetic construct
  misc_feature 1..3: note = RNA
  source 1..29: mol_type = other DNA
gcgaaaaaaaaaaaaaaaaaaaaaaaaaa

SEQ ID NO: 47 - DNA, 25 bp, synthetic construct
  misc_feature 1..3: note = RNA
  source 1..25: mol_type = other DNA
gcgaaaaaaaaaaaaaaaaaaaacg

SEQ ID NO: 48 - DNA, 26 bp, synthetic construct
  misc_feature 1..3: note = RNA
  source 1..26: mol_type = other DNA
gcgtaaaaaaaaaaaaaaaaaaaacg

SEQ ID NO: 49 - DNA, 26 bp, synthetic construct
  misc_feature 1..3: note = RNA
  source 1..26: mol_type = other DNA
gcgaaaaaaaaaaataaaaaaaaacg

SEQ ID NO: 50 - DNA, 30 bp, synthetic construct
  source 1..30: mol_type = other DNA
ttttttttttttcgcttagcctctgccttt

SEQ ID NO: 51 - DNA, 40 bp, synthetic construct
  source 1..40: mol_type = other DNA
tttttttttttttttttcgcgctaatcttttagcctctgc

SEQ ID NO: 52 - DNA, 40 bp, synthetic construct
  source 1..40: mol_type = other DNA
ttttttttttttttttacgcgctaatcttttagcctctgc

SEQ ID NO: 53 - DNA, 40 bp, synthetic construct
  source 1..40: mol_type = other DNA
tttttatttttttttttcgcgctaatcttttagcctctgc

SEQ ID NO: 54 - RNA, 71 nt, synthetic construct
  source 1..71: mol_type = other RNA
gggagagccaccatggtgagcggctggcggctgttcaagaagattagctaaaaaaaaaaaaaaaaaaaaaa

SEQ ID NO: 55 - DNA, 70 bp, synthetic construct
  misc_feature 1..48: note = RNA
  source 1..70: mol_type = other DNA
gggagagccaccatggtgagcggctggcggctgttcaagaagattagcaaaaaaaaaaaaaaaaaaaacg

SEQ ID NO: 56 - DNA, 36 bp, synthetic construct
  source 1..36: mol_type = other DNA
atggtgagcggctggcggctgttcaagaagattagc

(19) United States
(12) Patent Application Publication
(10) Pub. No.: US 2026/0226129 A1
(43) Pub. Date: Aug. 6, 2026

(54) ARTIFICIAL POLYNUCLEOTIDE MOLECULES FOR ENHANCED STABILITY AND TRANSLATION

(71) Applicant: ELEVEN THERAPEUTICS LTD, Ramat Gan (IL)

(72) Inventors: Yaniv ERLICH, Raanana (IL); Omer ZIV, Cambridge (GB); Andrew BEST, Cambridge (GB); Christine MORDSTEIN, Great Shelford (GB)

(21) Appl. No.: 19/151,523

(22) PCT Filed: Jan. 25, 2024
(86) PCT No.: PCT/EP2024/051723

§ 371 Date: Jul. 28, 2025

Related U.S. Application Data
(60) Provisional application No. 63/441,462, filed on Jan. 27, 2023.

(51) Int. Cl. C07K 14/605; A61K 38/00

(52) U.S. Cl. CPC C07K 14/605; A61K 38/00

(57) ABSTRACT

The present invention discloses artificial polynucleotide molecules comprising at least one open reading frame (ORF) and a synthetic 3′ terminus with increased stability and translational efficiency. The invention further discloses synthetic 3′ terminus compounds for linking to mRNA or mRNA-like molecules such as therapeutic mRNAs or replacing the conventional 3′ termini of said molecules. Further disclosed are methods for production and use as well as kits and pharmaceutical compositions comprising said artificial polynucleotide molecules and synthetic 3′ terminus compounds, preferably for use as therapeutic.

### FIELD OF THE INVENTION

The present invention relates to artificial polynucleotide molecules comprising at least one open reading frame (ORF) and a synthetic 3′ terminus with increased stability and/or translational efficiency as well as related therapeutic polynucleotide constructs encoding therapeutic polypeptides. The invention further relates to synthetic 3′ terminus compounds useful for linking to mRNAs or mRNA-like molecules such as therapeutic mRNAs or replacing conventional 3′ termini of said molecules. Further disclosed are methods for production and use as well as kits and pharmaceutical compositions comprising said artificial polynucleotide molecules, therapeutic polynucleotide constructs, and/or synthetic 3′ terminus compounds.

### BACKGROUND OF THE INVENTION

In recent years, there has been significant interest in using RNAs as therapeutic agents. RNA molecules can be designed to produce endogenous and foreign polypeptides and potentially compensate for insufficient production levels of specific proteins. Encoding mRNAs to produce specific therapeutics polypeptides, and induce an immunological memory against infectious agents has been demonstrated on a large scale in recent years.

However, one shortcoming of this modality is the relatively short duration of the effect of mRNAs due to its chemical instability and the existence of multiple RNA degrading enzymes inside cells, body fluids, and the environment more generally: RNA is much more unstable than DNA due to its chemical structure and RNA can be subject to autocatalysis as well as degradation by RNases, which are abundant in the environment due to their presence in sources such as sloughed-off skin cells and ubiquitous microorganisms.

The stability and translation efficiency of mRNAs is influenced by various factors. The structure of an mRNA molecule can be divided into several parts including a 5′ Cap, 3′ poly(A) tail, 5′ and 3′ untranslated regions (5′- and 3′-UTRs), and an open reading frame (ORF). A number of these components can be altered or modified to enhance the translatability and/or stability of mRNAs to make them suitable for therapeutic mRNA drugs.

In endogenous mRNAs, the 3′ UTR starts downstream of the open reading frame's translation stop-codon and ends with a string of adenine RNA nucleotides termed poly-(A) tail. Poly(A) tails are pivotal for the translation of mRNAs and lack thereof results in rapid degradation within the cell. The poly(A) tail also contributes to mRNA stability, and its length is positively correlated to translational efficiency (Wang, Y S., et al., J Biomed Sci 30, 84 2023). The poly(A) tail can be incorporated in the plasmid template, added via PCR, or added post-transcriptionally by enzymatic polyadenylation, which generates variable lengths of poly(A) tails. In mammalian cells, the poly(A) length is about 250 nt, but it is gradually reduced during an organism's lifetime. For mRNA drugs, it has been shown that a poly(A) tail length of approximately 100 nt is optimal to minimize decay (Schlake T, et al., RNA Biol. 2012 November; 9(11):1319-30. doi: 10.4161/rna.22269. Epub 2012 Oct. 12.). A segmented poly(A) approach of adding smaller spacer lengths between poly(A) segments in the DNA template can lead to higher translation efficiency and reduce plasmid recombination in E. coli, as compared to the use of conventional homogeneous poly(A) tails (Trepotec Z et al., RNA. 2019; 25:507-18.). While a poly(A) tail is seen as a requirement of a functional mRNA, the relatively long sequence increases the overall length of the mRNA, which can impact various properties of potential mRNA drugs due to the higher molecular weight and steric properties impacting deliverability.

The open reading frame (ORF) of mRNAs spans the RNA sequence between a START and STOP codon and may encode a polypeptide. The lack of a STOP codon can result in faulty protein production, reduced mRNA translation, stability, or degradation of the entire mRNA by Non-stop decay (NSD). NSD is a cellular mechanism of mRNA surveillance to detect mRNA molecules lacking a stop codon and prevent these mRNAs from translation. The non-stop decay pathway releases ribosomes that have reached the far 3′ end of an mRNA and guides the mRNA to the exosome complex, or to RNase R in bacteria for selective degradation. Accordingly, a STOP codon appears to be indispensable for RNA stability and translational efficiency of conventional mRNAs.

The accepted minimal requirements such as presence of a STOP codon within the ORF, or a poly(A) tail, for a functional mRNA limit the possible design options of scientists researching mRNA or mRNA-like molecules with preferable properties such as increased stability and enhanced translational efficiency. Accordingly, there is an intense need by the industry to overcome the minimal requirements of a functional mRNA as described above thereby providing a higher degree of freedom for mRNA design ultimately benefiting the development of, for instance, mRNA-based therapeutics.

Efforts have been made in recent years to improve chemical stability, translational efficiency and reduce immunogenicity of mRNAs by altering various components of canonical mRNAs. In the context of mRNA molecules, it is well known in the art that modifications to the uracil nucleobase, such as N1-methylpseudouridine and pseudouridine, are highly important to enhance the activity of the mRNA, reduce its inflammatory potential, and increase translation of the encoded protein or peptide.

However, the current landscape of potential chemical modifications for therapeutics mRNA is limited. As of today, all approved mRNA therapeutics are synthesized using in vitro transcription (IVT) reaction. In this process, a DNA-dependent RNA polymerase (DdRP), such as the bacteriophage T7 RNA polymerase, are incubated with template DNA molecules that encode the desired mRNA together with a mix of nucleoside-5-triphosphates. The DdRP scans the DNA molecule and polymerases the nucleoside-5-triphosphates into the mRNA molecule.

While being the main driver of mRNA therapeutics, IVT poses significant limitations regarding the possible chemical modifications to be incorporated. First, polymerization with DdRP enzymes is limited to certain types of modified nucleotides. For instance, it was found that even after extensive mutations to the T7 polymerases, it cannot easily polymerase 2′-OMe guanine nucleosides. Second, IVT is not amenable for site specific control over the placement of the chemical modification. For instance, in the case of using N1-methylpseudouridine nucleosides instead of uracil, all corresponding T positions in the template DNA will be polymerized as N1-methylpseudouridine in the mRNA molecule. However, it is well known that certain modifications are beneficial in a context dependent manner. For instance, eukaryotic mRNA can exhibit 2′-OMe modifications in the first two nucleotides from the 5′-end of the molecule, which can enhance translation and reduce immune response. However, fully modified 2′-OMe mRNA abolishes the translation activity. Finally, IVT does not allow for incorporation of non-nucleoside building blocks into the mRNA molecule, again limiting the potential chemical landscape.

Chemical synthesis of mRNA can overcome the limitations of IVT with respect to its chemical repertoire. Briefly, in one instance of this process, a specific type of 3′-phosphoramidite RNA nucleotides (e.g. A) are coupled into the 5′-hydroxyl end of the synthesized RNA molecules. The coupled nucleotides have on their 5′ end a protecting group to avoid uncontrolled coupling, which in some instances can be DMTr, an acid labile protecting group. After the coupling and other auxiliary steps, the synthesis apparatus is washed with acid to remove the protecting group and prepare the molecules for the next cycle. By washing the apparatus with modified phosphoramidite nucleotide, it is possible to incorporate it in a position-controlled manner. Similarly, many types of non-nucleotide building blocks can be incorporated as long as they can be incorporated to the exposed moiety after deprotection.

The main drawback of chemical synthesis is that the process is length limited. Owing to their 2′ hydroxyl, RNA molecules are base-labile and can undergo rapid hydrolysis even at mild base conditions. Protecting the 2′ hydroxyl can mitigate this issue but also diminish the coupling efficiency. In addition, the repeated acid washes can create depurination and abasic sites that can hamper the activity of the molecule.

To overcome this issue, it is also possible to employ a hybrid approach for the production of mRNA molecules. In this approach, certain parts of the mRNA molecules are produced by chemical synthesis, whereas the other parts are added using an enzymatic reaction. For example, Nagata et al. (NAR, 2010) chemically synthesized a 130 nt mRNA molecule with two phosphate groups (pyrophosphate) at the 5′ end. They added a m7GpppNm 5′ cap using an enzymatic capping system together with a 2′-O-methyltransferase and created a poly-A tail using a poly-A polymerase.

In addition, it is possible to overcome the length issue by stitching together mRNA elements using chemical and biological processes. For example, Walczak et al. (J. Chem Sci., 2017) have utilized copper-catalyzed azide-alkyne cycloaddition (CuAAC) to connect between mRNA with azide on its 5′ phosphate to m7Gppp with alkyne on its triphosphate. Alternatively, it is possible to conduct enzymatic ligation for instance by deploying T4 ligase and a splint between two or more RNA molecules as described in Kershaw et al. (Methods Mol. Bio., 2013).

While advances in recent years have improved the design and synthesis of therapeutic mRNAs, several challenges remain. The requirement of, for instance, a 3′UTR and poly(A) tail for efficient mRNA translation and stability dictate a minimum sequence length that can not only be difficult or uneconomically to produce but also interfere with delivery of the RNA to desired structures such as biological targets involved in disease. Additionally, while incorporation of modified nucleotides has increased RNA stability, mRNA molecules remain prone to degradation, therapeutically effective doses remain high, which can increase immunogenic reactions and increase costs.

### SUMMARY OF THE INVENTION

The objective of the present invention is to provide artificial polynucleotide molecules useful for applications as therapeutics. In particular, it is the objective to provide mRNA-like molecules with increased stability and/or translational efficiency compared to conventional mRNA molecules. Additionally, the objective is to provide cost-effectively synthetized mRNA-like constructs encoding, for instance, therapeutic polypeptides. A further aspect is to provide compounds that can be functionally linked to existing polynucleotides such as for instance mRNAs or mRNA fragments to improve stability and/or translational efficiency thereof in vitro and/or in vivo.

The technical problems underlying the present invention are solved by the claimed subject matter.

The present invention relates to an artificial polynucleotide molecule comprising or consisting of a polynucleotide M comprising or consisting of at least one open reading frame (ORF), and a synthetic 3′ terminus comprising or consisting of at least one polynucleotide P, wherein
the 3′ end of the polynucleotide M (i) is linked to the synthetic 3′ terminus (ii), and
said at least one polynucleotide P has a length of between about 4 nt and about 1000 nt, and comprises at least one DNA nucleotide, modified RNA, modified DNA or XNA nucleotide, non-natural backbone modification, modified nucleotide, modified backbone linkage, or any combination thereof.

In some embodiments the synthetic 3′ terminus (ii) increases stability and/or translational efficiency and/or bioavailability and/or reduces toxicity and/or immunogenicity of the artificial polynucleotide molecule.

In some embodiments, the artificial polynucleotide molecule is devoid of a canonical poly(A) tail. In some embodiments, the synthetic 3′ terminus (ii) is devoid of non-modified natural RNA nucleotides. In some embodiments, the polynucleotide M (i) is RNA. In some embodiments, the polynucleotide M (i) comprises at least one non-canonical RNA nucleotide.

In some embodiments, where the polynucleotide M does not comprise a 5′ CAP, the polynucleotide M (i) comprises at its 5′ end a removable protecting group selected from the group of levulinyl, [bis-(4-methoxyphenyl)phenylmethyl](DMTr), and fluorenylmethoxycarbonyl (FMOC). In a preferred embodiment, said protective group is DMTr.

In some embodiments, the polynucleotide M (i) further comprises at least one of structure (a) to (c), wherein
(a) denotes a 5′ CAP;
(b) denotes a 5′UTR; and
(c) denotes a 3′ UTR,
   or any combination thereof, and wherein said at least one structure is operably linked to the at least one ORF. In a preferred embodiment, the polynucleotide M (i), in an order of 5′ to 3′, comprises structures (a) to (c) operably linked to the at least one ORF. Preferably, said at least one structure is, in an order of 5′ to 3′, structure (a) and (b) operably linked to the at least one ORF and said artificial polynucleotide molecule is devoid of a canonical 3′UTR. In a preferred embodiment, said at least one structure is, in an order of 5′ to 3′, structure (b) operably linked to the at least one ORF and said artificial polynucleotide molecule is devoid of a canonical 3′UTR and 5′CAP.

In some embodiments the ORF of polynucleotide M is devoid of a STOP codon. In some embodiments, the at least one structure comprises, in order 5′ to 3′, 5′ CAP, 5′ UTR, ORF and 3′ UTR, wherein said ORF is devoid of STOP codon. In a preferred embodiment, the said at least one structure comprises, in order 5′ to 3′, 5′ CAP, 5′ UTR, and ORF, wherein said ORF is devoid of STOP codon. In some embodiments, the said at least one structure comprises, in order 5′ to 3′, 5′ UTR, and ORF, wherein said ORF is devoid of STOP codon. In some embodiments, where the polynucleotide M is devoid of a STOP codon, the ORF at the last codon comprises at least one modified nucleotide. In some embodiments, the ORF at the last codon comprises at least one DNA nucleotide. In some embodiments, the at least one DNA nucleotide is adenine nucleotide. In some embodiments, the last codon of the ORF consists of three adenine deoxynucleotides.

In some embodiments, the 3′ end of the polynucleotide M (i) is linked to the 5′ end of the at least one polynucleotide P of the synthetic 3′ terminus (ii).

In some embodiments, at least one ORF encodes at least one therapeutic polypeptide.

In some embodiments, the at least one polynucleotide P is of length between about 4 nt and about 1000 nt, preferably between about 4 nt and about 500 nt, preferably between about 4 nt and about 100 nt, preferably between about 4 nt and about 50 nt, and more preferably between about 4 nt and about 40 nt.

In some embodiments, the at least one polynucleotide P comprises or consists of at least 50%, preferably at least 60%, preferably at least 70%, preferably at least 80%, preferably at least 90%, preferably at least 95%, or most preferably 100% modified and/or unmodified DNA nucleotides.

In some embodiments, the said modified DNA nucleotides are selected from the group of DNA nucleotides with modifications to the ribose, modifications to the phosphate backbone, modifications to the nucleobases, stereoisomerization of the entire nucleotide, or any combination thereof. In some embodiments, the said modifications to the ribose are selected from the group of 2′-H (DNA), 2′-O-methoxy-ethyl, 2′-Fluoro, LNA, FANA, 2′-O-methyl, and PMO (morpholino). In some embodiments, the modification to the ribose is 2′-H(DNA).

In some embodiments, said modifications to the phosphate backbone are selected from the group of phosphonothioate links, Sp-only phosphonothioate links, Rp-only phosphonothioate links, phosphorodithioate links, peptide nucleic acid links, methylphosphonate links, ethoxypropylphosphonate links, exNA links, 3′-phosphonothiolate links, 5′-phosphonothiolate links or any combination thereof. In some embodiments, the modification to the phosphate backbone is phosphonothioate links.

In some embodiments, the modifications to the nucleobases are selected from the group of Biotin-labeled bases, N1-methyl-pseudo-uridine, pseudo-uridine, and 2,6-diaminopurine. In some embodiments, the modification to the nucleobase is Biotin addition.

In a preferred embodiment, the steroisomerization modified nucleotide is L-DNA.

In some embodiments, the artificial polynucleotide is a homopolymer of at least three identical modified nucleotides.

In some embodiments, the modified and/or unmodified DNA nucleotides comprises or consists of at least 50%, preferably at least 60%, preferably at least 70%, preferably at least 80%, preferably at least 90%, preferably at least 100% modified and/or unmodified deoxyadenosine (dA) nucleotides. In a preferred embodiment, the modified and/or unmodified DNA nucleotides comprises 100% modified deoxyadenosine nucleotides. In some embodiments, the modified dA nucleotides are linked to each nucleobase by a phosphorothioate bond.

In some embodiments, the at least one polynucleotide P is of length between about 10 nt and about 40 nt and comprises at least one Biotin-dT. In some embodiments, the polynucleotide P comprises or consists of, from 5′ to 3′, between 5 nt and 20 nt consecutive dA linked to a Biotin-dT linked to between 5 nt and 20 nt consecutive dA. In some embodiments, the at least one polynucleotide P comprises between about 10 to about 30, more preferably between 23 and 25 consecutive unmodified DNA nucleotides. In some embodiments, the at least one polynucleotide P, in an order of 5′ to 3′, comprises 25 consecutive unmodified DNA nucleotides followed by between about 3 and about 10, preferably between 3 and 5 consecutive modified DNA nucleotides.

In some embodiments, the synthetic 3′ terminus (ii) further comprises at least one building block selected from the group of L and C or any combination thereof, wherein L denotes a chemical linker and C denotes one or more monomers. In some embodiments, the building block L is selected from the group of non-RNA polymers, chemical spacers, doublers, sugars, lipids, peptides, aminonucleoside, vitamins, or any combination thereof. In some embodiments, the building block C is selected from the group consisting of GalNac, Biotin, DNA triplet, ACC-Puromycin, Cholesterol, C16, or any combination thereof. In some embodiments, the synthetic mRNA 3′ terminus (ii) further comprises at least one building block L. In a preferred embodiment, the at least one building block L denotes a chemical spacer Sp9. In some embodiments, the synthetic 3′ terminus (ii) comprises said polynucleotide P linked to 3 consecutive Sp9 spacers, and the 3′ end of said polynucleotide P is linked to the first Sp9 spacer.

In some embodiments, the synthetic 3′ terminus (ii) further comprises at least one building block C. In some embodiments, the at least one building block C is linked to the terminus of the third consecutive Sp9 spacer.

In some embodiments, the L and C building blocks are selected from elements that are resistant to digestion by endo- and/or exo-RNA and/or DNA nucleases. In some embodiments, the elements that are resistant to digestion by endo- and/or exo-RNA and/or DNA nucleases also facilitate delivery of nucleic acids into mammalian cells, including passive intake, receptor-mediated endocytosis, albumin binding, and endosomal release. In some embodiments, the elements that are resistant to digestion by endo- and/or exo-RNA and/or DNA nucleases are selected from the group of Sp9, GalNAc and L-dA.

In some embodiments, the artificial polynucleotide molecule is characterized by enhanced stability and/or translatability compared to artificial polynucleotide molecule according to the invention or to artificial polynucleotide molecule having a synthetic 3′ terminus consisting of L and C building blocks according to the invention. In some embodiments, the at least one building block C is ACC-Puromycin.

In some embodiments, the 5′ end of the at least one polynucleotide P is linked to the 3′ end of the polynucleotide M (i).

In some embodiments, the artificial polynucleotide molecule of the invention is according to formula (I):

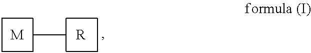

wherein M denotes a polynucleotide M (i) according to any of the preceding claims, and
R denotes a synthetic 3′ terminus (ii) of formula R0:

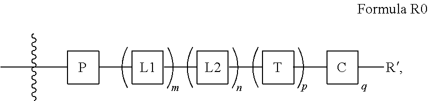

wherein
P denotes a polynucleotide P of the synthetic 3′terminus (ii) according to the invention,
L1 denotes a first building block L according to the invention,
L2 denotes a second building block L according to the invention,
T denotes a DNA triplet,
C denotes a building block C according to the invention,
m, n denote independently from each other an integer between 0 and 10,
p, q denotes independently from each 0 or 1,
R′ denote H, OH, canonical or, non-canonical nucleotide either individually or in conjunction with a delivery conjugate including but not limited to 5′GalNAc, CholTeG, Biotine
L1 and L2 are independently selected from the group of SpC3, Sp9, SpC12, Sp18, Spermine, 1-Ethynyl-dSpacer, CholTEG, L-dA, Bio-TEG linkers, and GalNAc,
L1 and L2 are different from each other and,
C is selected from the group of Puromycin, biotin-dT, 2′OC16-U, Beta-L-DNA, LNA, 2′Ome, 2′MOE, 2′F, Morpholino.

In a preferred embodiment R′ is H or OH, more preferably OH,

In some embodiments, R denotes a synthetic 3′ terminus selected from the group of formula L001 to L012:

| ID | Formula |
|---|---|
| L001 | 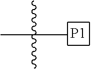 |
| L002 | 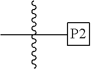 |
| L003 | 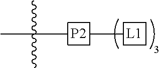 |
| L004 | 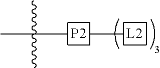 |
| L005 | 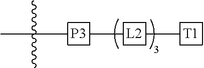 |
| L006 | 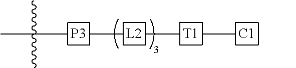 |
| L007 | 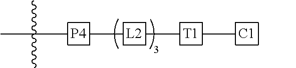 |
| L008 | 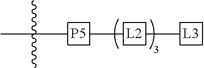 |
| L009 | 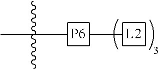 |
| L010 | 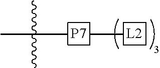 |
| L011 | 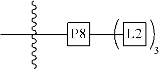 |
| L012 | 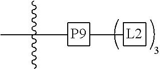 |

wherein
P1 denotes DNA polynucleotide of sequence AAAA,
P2 denotes a polynucleotide according to SEQ ID NO 1
P3 denotes a polynucleotide according to SEQ ID NO 2, wherein nucleobase at position 12 is biotinylated ([Bio-dT]),
P4 denotes a polynucleotide according to SEQ ID NO 3, wherein nucleobase at position 32 is biotinylated,
P5 denotes a polynucleotide according to SEQ ID NO 4, wherein nucleobases at position 21 and 22 are linked to the previous nucleobase by a phosphorothioate bond,
P6 denotes a polynucleotide according to SEQ ID NO 5, wherein nucleobase at position 15 is biotinylated ([Bio-dT]) and nucleobases at position 25 and 26 are linked to the previous nucleobase by a phosphorothioate bond,
P7 denotes a polynucleotide according to SEQ ID NO 6
P8 denotes a polynucleotide according to SEQ ID NO 7, wherein nucleobase at position 4 is biotinylated ([Bio-dT]) and nucleobases at position 25 and 26 are linked to the previous nucleobase by a phosphorothioate bond,
P9 denotes a polynucleotide according to SEQ ID NO 8 wherein nucleobases at position 24 and 25 are linked to the previous nucleobase by a phosphorothioate bond,
T1 denotes a DNA triplet of sequence ACC,
C1 denotes Puromycin,
L1 denotes L-dA,
L2 denotes Sp9,
L3 denotes C3 and
the 3′ end of said polynucleotide M is linked to the 5′ end of said synthetic 3′ terminus.

A further aspect of the invention relates to a synthetic 3′ terminus compound according to formula ST1 to ST10:

| ID | Formula |
|---|---|
| ST1 | 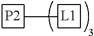 |
| ST2 | 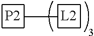 |
| ST3 | 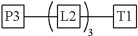 |
| ST4 | 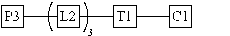 |
| ST5 | 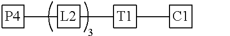 |
| ST6 | 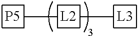 |
| ST7 | 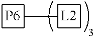 |
| ST8 | 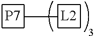 |
| ST9 | 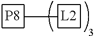 |
| ST10 | 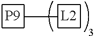 |

wherein
P2 denotes a polynucleotide according to SEQ ID NO 1
P3 denotes a polynucleotide according to SEQ ID NO 2, wherein nucleobase at position 12 is biotinylated ([Bio-dT]),
P4 denotes a polynucleotide according to SEQ ID NO 3, wherein nucleobase at position 32 is biotinylated,
P5 denotes a polynucleotide according to SEQ ID NO 4, wherein nucleobases at position 21 and 22 are linked to the previous nucleobase by a phosphorothioate bond,
P6 denotes a polynucleotide according to SEQ ID NO 5, wherein nucleobase at position 15 is biotinylated ([Bio-dT]) and nucleobases at position 25 and 26 are linked to the previous nucleobase by a phosphorothioate bond,
P7 denotes a polynucleotide according to SEQ ID NO 6
P8 denotes a polynucleotide according to SEQ ID NO 7, wherein nucleobase at position 4 is biotinylated ([Bio-dT]) and nucleobases at position 25 and 26 are linked to the previous nucleobase by a phosphorothioate bond,
P9 denotes a polynucleotide according to SEQ ID NO 8 wherein nucleobases at position 24 and 25 are linked to the previous nucleobase by a phosphorothioate bond,
T1 denotes a DNA triplet of sequence ACC,
C1 denotes Puromycin,
L1 denotes L-dA,
L2 denotes Sp9, and
L3 denotes C3.

In some embodiments, the 5′ end of the polynucleotide is operably linked to a RNA polynucleotide of length from about 1 nt to about 10 nt.

A further aspect of the invention relates to a method for the generation of the artificial polynucleotide molecule of the invention comprising or consisting of the steps:
providing a polynucleotide M (i) and a synthetic 3′ terminus (ii),
linking said polynucleotide M (i) to the synthetic 3′ terminus (ii), wherein the 3′ end of the polynucleotide M is linked to the synthetic 3′ terminus (ii).

A further aspect of the invention relates to a therapeutic polynucleotide construct comprising or consisting of the polynucleotide M (i), wherein the at least one ORF encodes at least one therapeutic polypeptide, and the synthetic 3′ terminus (ii) of the invention.

A further aspect of the invention relates to a pharmaceutical composition comprising the therapeutic polynucleotide of the invention.

In some embodiments, the pharmaceutical composition further comprises one or more pharmaceutically acceptable diluents and/or excipients and/or one or more adjuvants.

A further aspect of the invention relates to the artificial polynucleotide molecule, the therapeutic polynucleotide construct, or the pharmaceutical composition according to the invention for use as a medicament.

A further aspect of the invention relates to the artificial polynucleotide molecule, the therapeutic polynucleotide construct, or the pharmaceutical composition according to the invention for use as a vaccine.

A further aspect of the invention relates to a kit or kit of parts comprising the artificial polynucleotide molecule, the synthetic 3′ terminus of, the therapeutical polynucleotide construct, and/or the pharmaceutical composition according the invention.

A further aspect of the invention relates to method for obtaining a peptide or protein, comprising
providing an artificial polynucleotide molecule according the invention, and
translating said artificial nucleic acid.

A further aspect of the invention relates to the use of the artificial polynucleotide molecule of the invention for obtaining a peptide or protein.

A further aspect of the invention relates to the use of the artificial polynucleotide molecule of the invention for transfecting a host cell.

A further aspect of the invention relates to use of a synthetic 3′ terminus according to the invention for increasing stability and/or translational efficiency of a polynucleotide molecule, preferably of an mRNA molecule.

### Definitions

For convenience, certain terms employed in the specification, examples, and appended claims are collected here.

As used herein, the singular form “a,” “an,” and “the” include plural references unless the context clearly dictates otherwise. For example, the term “host cell” also includes a plurality of host cells.

The terms “polynucleotide” and “nucleic acid” are used herein interchangeably. They refer to a polymeric form of nucleotides of any length. Polynucleotides may have any three-dimensional structure, and may perform any function, known or unknown. The following are non-limiting examples of polynucleotides: coding or non-coding regions of a gene or gene fragment, exons, introns, messenger RNA (mRNA), transfer RNA, ribosomal RNA, ribozymes, cDNA, synthetic polynucleotides, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified, such as by conjugation with a labeling component. Polynucleotides depicted herein are in 5′ to 3′ direction unless otherwise stated.

If not stated otherwise, the term “nucleic acid” refers to any nucleic acid such as ribonucleic acid, deoxyribonucleic acid, xeno nucleic acid, single stranded or double stranded.

As used herein, the term “xeno nucleic acids” or “XNAs” are synthetic nucleic acid analogues that have a different phospho-sugar backbone or nucleobases than the natural nucleic acids DNA and RNA.

The term “RNA linker” according to the invention relates to a short RNA polynucleotide of length between 1 and 10 nt added between two nucleic acid sequences, such as between the polynucleotide M (i) and the polynucleotide P of the synthetic 3′ terminus (ii) according to the invention, to connect said two nucleic acid sequences. There is no limitation regarding the linker sequence.

The terms “codon” or “triplet” refers to a sequence of three nucleotides. A codon may encode an amino acid in a defined reading frame of a polynucleotide.

The term “operably linked” means with reference to a juxtaposition of two or more components (such as sequence elements), in which the components are arranged such that both components function normally and/or allow the possibility that at least one of the components can mediate a function that is exerted upon at least one of the other components.

The term “open reading frame” or “ORF” in the context of the invention may typically be a sequence of several nucleotide triplets which may be translated into a polypeptide or protein. An open reading frame preferably contains a start codon, i.e. a combination of three subsequent nucleotides coding usually for the amino acid methionine (ATG), at its 5′-end and a subsequent region which usually exhibits a length which is a multiple of 3 nucleotides. An ORF is preferably terminated by a stop-codon (e.g., TAA, TAG, TGA) unless stated otherwise. Typically, this is the only stop-codon of the open reading frame. Thus, an open reading frame in the context of the present invention is preferably a nucleotide sequence, consisting of a number of nucleotides that may be divided by three, which starts with a start codon (e.g. ATG) and which preferably terminates with a stop codon (e.g., TAA, TGA, or TAG). An ORF devoid of a STOP codon according to the invention relates to an ORF as described above wherein said ORF does not include any STOP-codon and said ORF is not terminated with a STOP-codon.

The term canonical or conventional “poly(A)”, or “poly(A) tail”, is typically understood to be a RNA sequence of adenine nucleotides, e.g., of up to about 400 adenine nucleotides, e.g. from about 11 to about 400. A poly(A) sequence is typically located at the 3′end of an mRNA. In the context of the present invention, an artificial polynucleotide molecule is devoid of a poly(A) sequence if said molecule does not comprise an RNA sequence of more than 10 uninterrupted adenine nucleotides downstream of its ORF.

The term “5′-untranslated region” or “5′UTR” refers to a nucleic acid sequence, which is typically part of an mRNA and is located between the 5′Cap and the open reading frame (ORF) of an mRNA. A 5′UTR of the mRNA is not translated into an amino acid sequence.

The term “3-untranslated region” or “3′UTR” refers to a nucleic acid sequence, which is typically part of an mRNA and is located between the open reading frame (ORF) and the poly(A) sequence of an mRNA. A 3′UTR of the mRNA is not translated into an amino acid sequence.

The term “5′Cap” refers to a small molecule at the 5′ end of the mRNA molecule that promotes canonical or non-canonical translation. In preferred embodiments the 5′Cap specifically binds one or to one or more translation initiation factors. In a more preferred embodiment, the 5′Cap specifically binds to eukaryotic translation initiation factor 4E (eIF4E). The term 5′Cap may also refer to one or more non-naturally chemically stabilizing elements located upstream of the opening reading frame of the RNA. Importantly, these one or more elements facilitate translation of a polypeptide from the mRNA despite the absence of a natural cap structure.

A 5′Cap may typically be formed by a modified nucleotide, particularly by a derivative of a guanine nucleotide. Preferably, the 5′Cap is linked to the 5′-terminus via a 5′-5′-triphosphate linkage. A 5′Cap may be methylated, e.g. m7GpppN, wherein N is the terminal 5′ nucleotide of the nucleic acid carrying the 5′Cap, typically the 5′-end of an RNA. Further non-limiting examples of 5′Cap structures include glyceryl, inverted deoxy abasic residue (moiety), 4′,5′ methylene nucleotide, 1-(beta-D-erythrofuranosyl) nucleotide, 4′-thio nucleotide, carbocyclic nucleotide, 1,5-anhydrohexitol nucleotide, L-nucleotides, alpha-nucleotide, modified base nucleotide, threo-pentofuranosyl nucleotide, acyclic 3′,4′-seco nucleotide, acyclic 3,4-dihydroxybutyl nucleotide, acyclic 3,5 dihydroxypentyl nucleotide, 3′-3′-inverted nucleotide moiety, 3′-3′-inverted abasic moiety, 3′-2′-inverted nucleotide moiety, 3′-2′-inverted abasic moiety, 1,4-butanediol phosphate, 3′-phosphoramidate, hexylphosphate, aminohexyl phosphate, 3′-phosphate, 3′phosphorothioate, phosphorodithioate, or bridging or non-bridging methylphosphonate moiety.

The term “translational efficiency” relates to a biological property of mRNAs or mRNA-like molecules, wherein the efficiency of polypeptide synthesis through translation of the mRNA or mRNA-like molecule is quantified. In particular, the term “translation efficiency” relates to the amount of translation product provided by an mRNA or mRNA-like molecule within a particular period of time. It is well known in the art how to quantify the amount of translation product. For instance, quantities of reporter protein encoded by the respective polynucleotide, such as mRNA, may be determined, e.g., by ELISA assays or reporter assays such as luciferase assays depending on the reporter protein used.

The term “stability” of a compound refers to the chemical and/or enzymatic stability of compounds such as mRNAs in vitro and/or in vivo. For instance, the stability of an mRNA can relate to the stability during storage, within solution, within the human body, or within a cell, or within a certain organelle in the cell, such as the endosome. Preferably, the term “stability of a polynucleotide” relates to the “half-life” of a polynucleotide. “Half-life” relates to the period which is needed to eliminate half of the activity, amount, or number of molecules. In the context of the present invention, the half-life of a polynucleotide or a molecule is indicative for the stability of said molecule. It is well established in the art how to determine the half-life of a polynucleotide, for example, the quantities of polynucleotide present in cells at the sample time points may be determined by quantitative PCR methods.

Preferably, the term “increased stability of the synthetic polynucleotide molecule” means that the half-life, e.g. the period of time which is needed to eliminate half of the activity, amount, or number of molecules, of the synthetic polynucleotide molecule of the invention after application (e.g. transfection into cell line or injection into a subject), is increased compared to the half-life of a reference mRNA.

The terms “increased translational efficiency”, “polypeptide production duration”, “polypeptide expression”, or “total polypeptide production” mean that polypeptide production from the artificial polynucleotide molecule of the invention is stabilized and/or prolonged compared to the polypeptide production from a reference nucleic acid such as a reference mRNA, e.g. comprising a comparable, preferably identical, polynucleotide M (i) but lacking said synthetic 3′ terminus (ii) of the invention, preferably in a mammalian expression system, such as an animal or cell culture system.

The term “biological activity” refers to a biological property such as pharmacological activity, efficacy or any other beneficial or adverse effects of a compound on living matter.

The term “delivery reagent” unless otherwise specified refers to any vehicle useful for the delivery of compounds to a tissue, cell or environment of choice. Non-limiting examples of delivery vehicles are nanoparticles, polymers, lipids, small molecules, aptamers, antibodies, and cell penetrating peptides, as well as any other vehicle known by the skilled person in the art.

The terms “polypeptide”, “peptide”, and “protein” relate to oligo- and polypeptides and refers to substances which comprise two or more, preferably 3 or more, preferably 4 or more, preferably 6 or more, preferably 8 or more, preferably 10 or more consecutive amino acids linked to one another via peptide bonds. The term “protein” refers to large peptides, preferably peptides having at least 151 amino acids, but the terms “(poly)peptide” and “protein” are used herein usually as synonyms. Polypeptides may further comprise according to the invention substances which contain not only amino acid components but also non-amino acid components such as sugars and phosphate structures, as well as substances containing bonds such as ester, thioether or disulfide bonds. According to the present invention, a nucleic acid such as RNA may encode a polypeptide or protein. Accordingly, a transcribable polynucleotide or a transcript thereof such as mRNAs may contain an open reading frame (ORF) encoding a polypeptide. Said polynucleotide may express the encoded polypeptide or protein. For example, said polynucleotide may be a nucleic acid encoding a therapeutical polypeptide.

The term “therapeutical polypeptide” relates to a polypeptide with a positive or advantageous effect on the condition or disease state of a subject when administered to the subject in a therapeutically effective amount. Preferably, a therapeutical polypeptide has curative or palliative properties and may be administered to ameliorate, relieve, alleviate, reverse, delay onset of or lessen the severity of one or more symptoms of a disease or disorder. A therapeutical polypeptide may have prophylactic properties and may be used to delay the onset of a disease or to lessen the severity of such disease or pathological condition. The term “therapeutical polypeptide” includes entire proteins or polypeptides, and can also refer to pharmaceutically active fragments thereof. It can also include pharmaceutically active analogs of a peptide or protein. The term “therapeutical polypeptide” includes polypeptides and proteins that are antigens, i.e., the polypeptide or protein elicits an immune response in a subject which may be therapeutic or partially or fully protective.

Examples of therapeutical polypeptides include, but are not limited to, cytokines and immune system proteins such as immunologically active compounds (e.g., interleukins, colony stimulating factor (CSF), granulocyte colony stimulating factor (G-CSF), granulocyte-macrophage colony stimulating factor (GM-CSF), erythropoietin, tumor necrosis factor (TNF) , interferons, integrins, addressins, seletins, homing receptors, T cell receptors, immunoglobulins, soluble major histocompatibility complex antigens, immunologically active antigens such as bacterial, parasitic, or viral antigens, allergens, autoantigens, antibodies), hormones (insulin, thyroid hormone, catecholamines, gonadotrophines, trophic hormones, prolactin, oxytocin, dopamine, bovine somatotropin, leptins and the like), growth hormones (e.g., human grown hormone), growth factors (e.g., epidermal growth factor, nerve growth factor, insulin-like growth factor and the like), growth factor receptors, enzymes (tissue plasminogen activator, streptokinase, cholesterol biosynthetic or degradative, steriodogenic enzymes, kinases, phosphodiesterases, methylases, de-methylases, dehydrogenases, cellulases, proteases, lipases, phospholipases, aromatases, cytochromes, adenylate or guanylaste cyclases, neuramidases and the like), receptors (steroid hormone receptors, peptide receptors), binding proteins (growth hormone or growth factor binding proteins and the like), transcription and translation factors, tumor growth suppressing proteins (e.g., proteins which inhibit angiogenesis), structural proteins (such as collagen, fibroin, fibrinogen, elastin, tubulin, actin, and myosin), blood proteins (thrombin, serum albumin, Factor VII, Factor VIII, insulin, Factor IX, Factor X, tissue plasminogen activator, protein C, von Willebrand factor, antithrombin Ill, glucocerebrosidase, erythropoietin granulocyte colony stimulating factor (GCSF) or modified Factor VIII, anticoagulants and the like.

As used herein “therapeutically effective amount” refers to an amount of a composition that relieves (to some extent, as judged by a skilled medical practitioner) one or more symptoms of the disease or condition in a mammal. Additionally, by “therapeutically effective amount” of a composition is meant an amount that returns to normal, either partially or completely, physiological or biochemical parameters associated with or causative of a disease or condition. A clinician skilled in the art can determine the therapeutically effective amount of a composition in order to treat or prevent a particular disease condition, or disorder when it is administered, such as intravenously, subcutaneously, intraperitoneally, orally, or through inhalation. The precise amount of the composition required to be therapeutically effective will depend upon numerous factors, e.g., such as the specific activity of the active agent, the delivery device employed, physical characteristics of the agent, purpose for the administration, in addition to many patient-specific considerations. But a determination of a therapeutically effective amount is within the skill of an ordinarily skilled clinician upon the appreciation of the disclosure set forth herein.

The terms “treating,” “treatment,” “therapy,” and “therapeutic treatment” as used herein refer to curative therapy, prophylactic therapy, or preventative therapy. An example of “preventative therapy” is the prevention or lessening the chance of a targeted disease (e.g., cancer or other proliferative disease) or related condition thereto. Those in need of treatment include those already with the disease or condition as well as those prone to have the disease or condition to be prevented. The terms “treating,” “treatment,” “therapy,” and “therapeutic treatment” as used herein also describe the management and care of a mammal for the purpose of combating a disease, or related condition, and includes the administration of a composition to alleviate the symptoms, side effects, or other complications of the disease, condition. Therapeutic treatment for cancer includes, but is not limited to, surgery, chemotherapy, radiation therapy, gene therapy, and immunotherapy.

Unless defined otherwise, the technical and scientific terms used herein have the same meaning as commonly understood by one of ordinary skill in the art to which the invention belongs. Particularly, unless otherwise stated, a term as used herein is given the definition as provided in the Oxford dictionary of biochemistry and molecular biology, Oxford University Press, 1997, revised 2000 and reprinted 2003, ISBN 0 19 850673 2.

### DETAILED DESCRIPTION OF THE INVENTION

A first aspect of the present invention refers to an artificial polynucleotide molecule comprising or consisting of
(i) a polynucleotide M comprising or consisting of at least one open reading frame (ORF), and
(ii) a synthetic 3′ terminus comprising or consisting of at least one polynucleotide P.

Surprisingly, it was found that the artificial polynucleotide molecule according to the invention represents or provides an mRNA/mRNA-like molecule with increased chemical and/or enzymatic stability compared to conventional mRNA. Additionally, it was found that the artificial polynucleotide molecule according to the invention increases the translational efficiency thereby allowing for prolonged and/or stabilized polypeptide production. Thus, the artificial polynucleotide molecule as described herein provides a highly stable molecule with prolonged polypeptide production in vitro and in vivo compared to a canonical/conventional mRNA molecule encoding a comparable or the same open reading frame.

Preferably, the at least one open reading frame (ORF) of the polynucleotide M (i) is functionally linked to the synthetic 3′ terminus (ii). This means that preferably the synthetic 3′ terminus (ii) is associated with the at least one ORF such that it may exert a function, such as a stabilizing function on the translation of a polypeptide encoded by the at least one ORF or a stabilizing function on the artificial polynucleotide molecule. Preferably, the polynucleotide M (i) and the synthetic 3′ terminus (ii) are associated in 5′ to 3′ direction. Thus, preferably, the artificial polynucleotide molecule comprises in direction of 5′ to 3′ structures ORF (i), an optional RNA linker, and the synthetic 3′ terminus (ii), wherein the RNA linker may be present or absent.

In some embodiments, the synthetic 3′ terminus of the artificial polynucleotide molecule according to the invention increases the translational efficiency, polypeptide production duration, polypeptide expression, total polypeptide production, or any combination thereof, of the artificial polynucleotide molecule. In some embodiments, the synthetic 3′ terminus of the artificial polynucleotide molecule according to the invention increases the stability of the artificial polynucleotide molecule. More preferably, the synthetic 3′ terminus of the artificial polynucleotide molecule according to the invention increases stability and/or translational efficiency.

In a preferred embodiment of the present invention, the synthetic 3′ terminus (ii) fulfills at least one function selected from the group of increasing the chemical and/or enzymatical stability and enhancing the translational efficiency of the polynucleotide M (i), preferably in a mammalian cell, such as but not limited to a human cell, compared to a nucleic acid, such as a canonical mRNA, comprising said at least one ORF of polynucleotide M (i) of the present invention. A canonical mRNA in this context may be, for instance, an mRNA naturally occurring comprising said at least one ORF of polynucleotide M (i).

Said increase in stability, translational efficiency, polypeptide production duration, polypeptide expression, or total polypeptide production is preferably determined by comparison with a respective reference polynucleotide molecule (reference mRNA) lacking the synthetic 3′ terminus according to the invention, e.g. an mRNA lacking a 3′ terminus, or a reference mRNA comprising a reference 3′ terminus, such as a 3′UTR and/or poly(A) sequence naturally occurring with the ORF as describe above.

A reference mRNA/polynucleotide in this context means that the reference mRNA/polynucleotide comprises a comparable, preferably identical, polynucleotide M (i) of the artificial polynucleotide molecule but does not comprise the synthetic 3′ terminus (ii) according to the invention.

Accordingly, polypeptide production from the artificial polynucleotide molecule according to the present invention is observable for a longer period of time compared to what is observable, or would be observable, from a reference mRNA or canonical mRNA. In other words, the amount of polypeptide produced from the artificial polynucleotide molecule of the invention measured over time is larger than the amount of polypeptide produced from a reference mRNA measured over the same time and under comparable conditions. For example, the amount of polypeptide production of the artificial polynucleotide molecule of the invention measured in the initial phase of expression, such as 1 h, 2 h, 3 h, 4 h, 5 h, or 6 h, or during a prolonged period of time, such as 12 h, 24 h, 48 h, 72 h post administration of the polynucleotide molecule is larger than the amount measured under the same time and conditions for a reference mRNA.

The increase of stability and/or translational efficiency may be determined by any method suitable for this purpose known to skilled person. For example, reference mRNAs may be generated comprising a coding sequence for a reporter protein, such as luciferase, and no 3′UTR, a 3′UTR derived from a reference gene (i.e., a reference 3′UTR, such as a 3′UTR naturally occurring with the ORF), and/or a canonical poly(A) sequence. Such mRNAs may be generated, for example, by in vitro transcription of respective vectors such as plasmid vectors, e.g. comprising a T7 promoter and a sequence encoding the respective mRNA sequences. The generated mRNA molecules may be transfected into cells by any transfection method suitable for transfecting mRNA, for example they may be electroporated into mammalian cells, such as A549 or HEK 293T cells, and samples may be analyzed at certain time points after transfection, for example, 6 hours, 24 hours, 48 hours, and 72 hours post transfection.

Preferably, the at least one function exerted by the synthetic 3′ terminus according to the invention, such as increasing stability and/or enhancing translational efficiency of the polynucleotide M (i), is at least 10%, more preferably at least 20%, more preferably at least 30%, more preferably at least 40%, more preferably at least 50%, more preferably at least 60%, more preferably at least 80%, more preferably at least 90%, and even more preferably at least 100% increased, compared to a nucleic acid, such as a canonical mRNA, comprising said polynucleotide M (i) of the present invention, as preferably measured 48 h post transfection.

### Polynucleotide M (i)

The polynucleotide M (i) and the synthetic 3′ terminus (ii) are heterologous, wherein heterologous in this context means that the polynucleotide M (i) and the synthetic 3′ terminus (ii) of the artificial polynucleotide molecule according to the invention are not occurring naturally in this combination.

In some embodiments, the polynucleotide M (i) is RNA. The artificial polynucleotide molecule according to the present invention may further comprise optional structures, commonly found in canonical mRNAs, including but not limited to a 5′UTR, 3′UTR and/or a 5′-Cap. Said optional structures are preferably operably linked to the at least one ORF of the polynucleotide M (i). For instance, the optional 5′-Cap and/or the 5′UTR are preferably located 5′ or upstream to the ORF and the optional 3′UTR 3′ or downstream to the ORF within the artificial polynucleotide molecule according to the present invention. In some embodiments the structure of the artificial polynucleotide molecule according to the invention is, from 5′ to 3′, 5′Cap-5′UTR-ORF-3′UTR-synthetic 3′ terminus. In another embodiment the artificial polynucleotide molecule according to the invention is, from 5′ to 3′, 5′cap-5′UTR-ORF-synthetic 3′ terminus.

A 5′UTR useful according to the present invention may refer to a non-coding regulatory element linked to the at least one ORF of the polynucleotide M (i) of the invention. 5′UTRs can be derived from the original 5′UTR associated with the ORF as seen in nature. However, in a preferred embodiment 5′UTRs useful according to the present invention are short naturally occurring or synthetic polynucleotide sequences. In some embodiment 5′UTRs have a length of about 10,000 monomers to about 1 monomer, more preferred a length of about 1,000 monomers to about 1 monomer, more preferred of about 100 monomers to about 5 monomers and most preferred of about 50 monomers to about 5 monomers.

3′UTRs useful according to the present invention are short naturally occurring or synthetic polynucleotide sequences. In some embodiment 3′UTRs have a length of about 10,000 monomers to about 1 monomer, more preferred a length of about 1,000 monomers to about 1 monomer, more preferred of about 100 monomers to about 5 monomers and most preferred of about 50 monomers to about 5 monomers.

The presence of a poly(A)-tail in canonical/conventional mRNA molecules is pivotal for stability and/or translational efficiency. Surprisingly, stability and/or translational efficiency of the artificial polynucleotide molecule according to the invention is significantly improved even in said molecules that are devoid of a canonical poly(A)-tail compared to a reference mRNA molecule comprising a canonical poly(A)-tail and comprising the same at least one ORF of the polynucleotide M (i) according to the invention. Accordingly, the synthetic 3′ terminus (ii) of the artificial polynucleotide molecule of the invention is not only able to replace canonical poly(A)-tails found in conventional mRNAs but also increase enzymatical/chemical stability and/or translational efficiency resulting in prolonged polypeptide production. Therefore, in a preferred embodiment, the artificial nucleic molecule is devoid of a canonical poly(A) tail.

The presence of a STOP codon at the 3′ end of an ORF comprising a conventional mRNA is indispensable for translational efficiency, wherein a lack of such codon regularly results, for instance, in non-stop decay and thus removal of the mRNA. Surprisingly, the lack of a STOP codon at the 3′ terminus of the at least one ORF and/or the artificial polynucleotide molecule according to the invention resulted in strong polypeptide production demonstrating that a STOP codon may be dispensable for polypeptide expression. Accordingly, in some embodiments the at least one ORF comprising the artificial polynucleotide molecule of the invention is devoid of a STOP codon.

Surprisingly, the lack of a 3′UTR within the artificial polynucleotide molecule according to the invention resulted in strong protein expression demonstrating that a 3′UTR may be further dispensable for polypeptide expression. Accordingly, in some embodiments the artificial nucleic acid molecule of the invention is devoid of a 3′UTR.

In a preferred embodiment, the at least one ORF comprising the polynucleotide M according to the invention encodes at least one therapeutic polypeptide. A therapeutical polypeptide according to the present invention relates to any polypeptide useful for the treatment of subjects in need thereof.

### Synthetic 3′ Terminus (ii)

The synthetic 3′ terminus (ii) according to the invention refers to a molecule comprising or consisting of at least one polynucleotide P and optionally at least one building block L and/or at least one building block C, or any combination thereof, wherein L denotes a chemical linker and C denotes a at least one monomer.

In a preferred embodiment, synthetic 3′ terminus (ii) according to the invention is devoid of non-modified natural RNA nucleotides.

### Polynucleotide P

In some embodiments the at least one polynucleotide P according to the invention is of length between about 4 nt and about 1000 nt, preferably between 4 nt and 500 nt, preferably between 4 nt and 400 nt, preferably between 4 nt and 300 nt, preferably between 4 nt and 200 nt, preferably between 4 nt and 100 nt, more preferably between about 4 nt and about 60 nt, more preferably between about 4 nt and about 50 nt, more preferably between about 4 nt and about 40 nt, more preferably between about 10 nt and about 40 nt, more preferably between about 20 nt and about 60 nt, and preferably between about 25 nt and about 35 nt.

In some embodiments the at least one polynucleotide P of the synthetic 3′ terminus according to the invention comprises at least one DNA nucleotide, modified RNA, DNA or XNA nucleotide, non-natural backbone modification, modified nucleotide, modified backbone linkage, or any combination thereof.

In some embodiments the at least one polynucleotide P of the invention comprises at least 50%, preferably at least 60%, preferably at least 70%, preferably at least 80%, preferably at least 90%, preferably at least 95%, or most preferably 100% modified and/or unmodified DNA nucleotides.

The selection of nucleobases determining the sequence of said at least one polynucleotide P can significantly influence the properties of the artificial polynucleotide molecule according to the invention. Surprisingly, it has been found that higher proportions of dA nucleotides within the polynucleotide P can further increase stability and efficiency. Accordingly, in some embodiments, said modified and/or unmodified DNA nucleotides are at least 50%, preferably at least 60%, preferably at least 70%, preferably at least 80%, preferably at least 90% modified and/or unmodified dA nucleotides. In some embodiments, said modified and/or unmodified DNA nucleotides are 100% dA.

The incorporation of modified nucleotides into the at least one polynucleotide P may increase translational efficiency and stability even further. Accordingly, in some embodiments said modified DNA nucleotides are selected from the group of modifications to the ribose, modifications to the phosphate backbone, modifications to the nucleobases, or any combination thereof. In some embodiments, said modifications to the ribose are selected from the group of L-ribose, 2′-F, and 2′-O-methyl. In some embodiments said modifications to the phosphate backbone are selected from the group of phosphonothioate links, 3′-phosphonothiolate links, or 5′-phosphonothiolate links. In some embodiments said modifications to the nucleobases are selected from the group of N1-methyl-pseudo-uridine, pseudo-uridine, Biotin-dT, and 2,6-diaminopurine.

In some embodiments, the at least one polynucleotide P comprises between about 1 to about 500, more preferably between 4 and 100, more preferably between 10 and 50, more preferably between 15 and 40, more preferably between 18 and 35, more preferably between 20 and 30, more preferably between 20 and 25, and even more preferably between 23 and 25 consecutive unmodified DNA nucleotides.

In some embodiment, said at least one polynucleotide P comprises, from 5′ to 3′, between 5 nt and 50 nt, preferably between 10 nt and 30 nt, more preferably between 20 nt and 25 nt, more preferably between 23 nt and 25 nt consecutive unmodified dA linked to at least one modified nucleotide linked to between 5 nt and 50 nt, preferably between 10 nt and 30 nt, more preferably between 20 nt and 25 nt, more preferably between 23 nt and 25 nt consecutive unmodified dA.

In a preferred embodiment, the at least one polynucleotide P comprises at least one Biotin-dT. In a more preferred embodiment, said at least one polynucleotide P comprises, from 5′ to 3′, between 5 nt and 20 nt consecutive dA linked to a Biotin-dT linked to between 5 nt and 20 nt consecutive dA.

### Building Block L

In some embodiments, the artificial polynucleotide molecule further comprises at least one building block L, wherein L denotes a chemical linker. In some embodiments, said building block L is selected from the group of non-RNA polymers, chemical spacers, doublers, sugars, lipids, peptides, aminonucleoside, vitamins, or any combination thereof. In a preferred embodiment, said at least one building block L is selected from the group of SpC3, Sp9, SpC12, Sp18, Spermine, 1-Ethynyl-dSpacer, CholTEG, L-dA, Bio-TEG linkers, and GalNAc. In a preferred embodiment, said at least one building block L is a chemical spacer, more preferably a Sp9 spacer. In a preferred embodiment, the artificial polynucleotide molecule further comprises three consecutive Sp9 spacers. In a more preferred embodiment, said at least one polynucleotide P is linked to three consecutive Sp9 spacers, wherein the 3′ end of said polynucleotide P is linked to the first Sp9 spacer.

### Building Block C

In some embodiments, the artificial polynucleotide molecule further comprises at least one building block C, wherein C denotes at least one monomer/small molecule conjugate. In a preferred embodiment, said at least one building block C is selected from the group consisting of Puromycin, biotin-dT, 2′OC16-U, Beta-L-DNA, LNA, 2′Ome, 2′MOE, 2′F, and Morpholino. In some embodiments, said at least one building block C is Puromycin. In a more preferred embodiment, said at least one building block C is Puromycin linked to between 1 and 10 nucleotides, more preferably to between 2 and 5 nucleotides, and most preferably to 3 nucleotides. In some embodiments said at least one building block C is Puromycin linked to a polynucleotide of any sequence, preferably of sequence, in 5′ to 3′ direction, ACC, wherein the 3′ end of the polynucleotide of sequence ACC is linked to a puromycin molecule (ACC-Puromycin).

Surprisingly, the incorporation of building blocks L and/or C within the synthetic 3′ terminus (ii) according to the invention has a synergistic effect on stability of the artificial polynucleotide molecule of the invention. The structural framework of the monomers/building blocks plays a pivotal role. Each monomer, carefully selected for its specific properties, contributes to the overall construction of the synthesized sequence. The chosen monomers exhibit a characteristic phosphodiester linkage, a fundamental feature in the formation of nucleic acid chains. This linkage involves the condensation of the hydroxyl (—OH) group of one monomer with the 5′ phosphate group of another, resulting in the creation of a robust covalent bond.

All building blocks (building blocks L and C) described herein are well-known non-standard monomer units of defined structure and correspond to polynucleotide synthesis nomenclature. (See e.g., the web-site of Integrated DNA Technologies (IDT) at https://eu.idtdna.com/site/catalog/Modifications/GetAllMods, or GenLink at http://www.genelink.com/newsite/products/OligoModifications.asp for further details of commonly used polynucleotide nomenclature.) While it is well known how to link said building blocks to a polynucleotide and/or each other, exemplary illustrations can be found in Figure FIG. 13.

In some embodiments, the artificial polynucleotide molecule according to the invention comprises or consists of a compound according to formula (I):

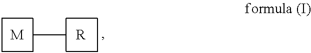

wherein M denotes a polynucleotide M (i) according to the invention, and
R denotes a synthetic 3′ terminus (ii) according to the invention of formula R0:

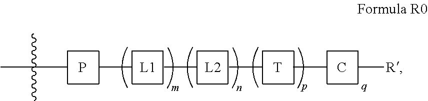

wherein
P denotes a polynucleotide P of the synthetic 3′terminus (ii) according to the invention,
L1 denotes a first building block L according to the invention,
L2 denotes a second building block L according to the invention,
T denotes a DNA triplet,
C denotes a building block C according to the invention,
m, n denote independently from each other an integer between 0 and 10,
p, q denotes independently from each 0 or 1,
R′ denote H, OH, canonical or, non-canonical nucleotide either individually or in conjunction with a delivery conjugate including but not limited to 5′GalNAc, CholTeG, Biotine,
L1 and L2 are independently selected from the group of SpC3, Sp9, SpC12, Sp18, Spermine, 1-Ethynyl-dSpacer, CholTEG, L-dA, Bio-TEG linkers, and GalNAc,
L1 and L2 are different from each other and,
C is selected from the group of Puromycin, biotin-dT, 2′OC16-U, Beta-L-DNA, LNA, 2′Ome, 2′MOE, 2′F, Morpholino.

In a preferred embodiment R′ is H or OH, more preferably OH.

In some embodiments, C is selected from a monomer chain of between 2 and 10 monomers selected from the group of Puromycin, biotin-dT, 2′OC16-U, Beta-L-DNA, LNA, 2′Ome, 2′MOE, 2′F, Morpholino. In a preferred embodiment, P is a polynucleotide according to SEQ ID NO i to 8.

The polynucleotide P of the Formula R0 is depicted in 5′ to 3′ direction, wherein the 5′ end of the first building block is linked to the 3′ end of the polynucleotide P. Each building block is linked to the previous building block according to the polynucleotide synthesis nomenclature from 5′ to 3′ by e.g., phosphodiester bonds (unless otherwise stated or evident according to the nomenclature) as known in the arts. While all building blocks indicated are known and well defined, the structure of a selected non-limiting set of building blocks useful according to the invention is illustrated below.

TABLE 1
Building block L

| Building | |
|---|---|
| block | Formula |
| SpC3 | 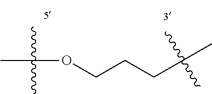 [SMILES: *CCCO*] |
| Sp9 | 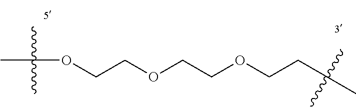 [SMILES: *CCOCCOCCO*] |
| SpC12 | 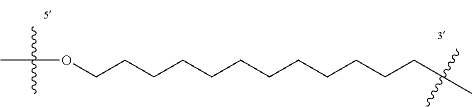 [SMILES: *CCCCCCCCCCCCO*] |
| Sp18 | 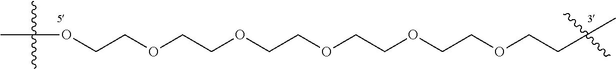 [SMILES: *CCOCCOCCOCCOCCOCCO*] |
| Spermine | 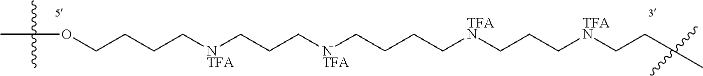 |
| 1-Ethynyl- dSpacer | 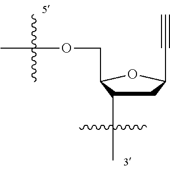 |
| CholTEG | 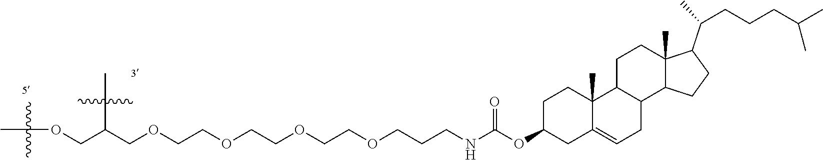 [SMILES: *OCC(*)COCCOCCOCCOCCCNC(=O)O[C@H]1CC[C@@]2(C)C(=CCC3C4CCC([C@H](C)CCCC(C)C)[C@@]4(C)CCC32)C1] |
| Bio-TEG | 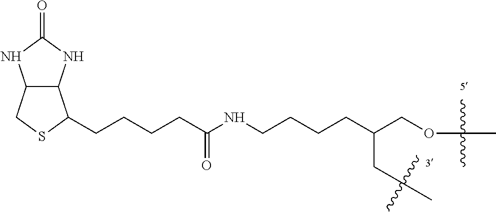 [SMILES: *CC(CCCCNC(=O)CCCCC1SCC2NC(=O)NC21)CO*] |
| GalNAc (3′) | 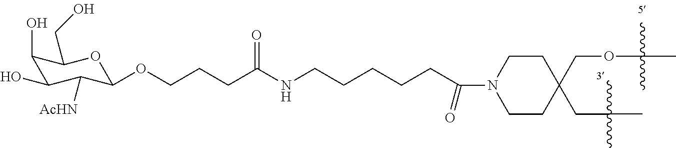 [SMILES: *CC1(CO*)CCN(C(=O)CCCCCNC(=O)CCCOC2OC(CO)C(O)C(O)C2NC(C)=O)CC1] |
| GalNAc (5′) | 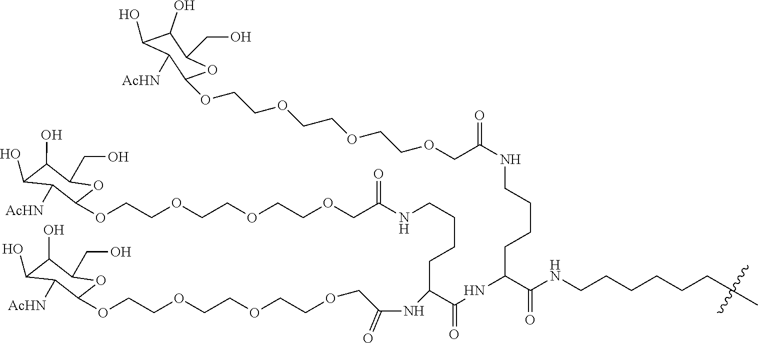 [SMILES: *CCCCCCNC(=O)C(CCCCNC(=O)COCCOCCOCCOC1OC(CO)C(O)C(O)C1NC(C)=O)NC(=O)C(CCCCNC(=O)COCCOCCOCCOC1OC(CO)C(O)C(O)C1NC(C)=O)NC(=O)COCCOCCOCCOC1OC(CO)C(O)C(O)C1NC(C)=O] |

TABLE 2
Building blocks C

| | |
|---|---|
| Puromycin | 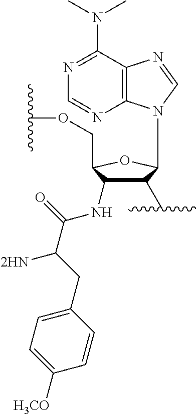 |
| Biotin-dT | 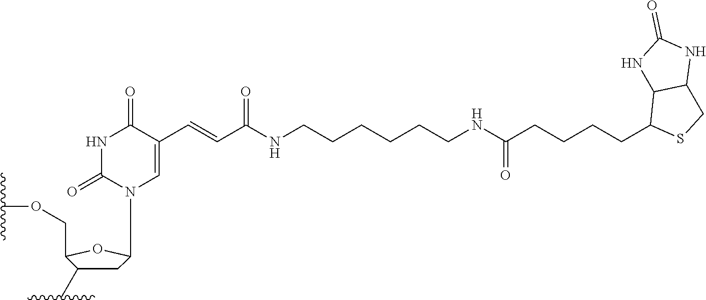 [SMILES: *OCC1OC(n2cc(/C=C/C(=O)NCCCCCCNC(=O)CCCCC3SCC4NC(=O)NC43)c(=O)[nH]c2=O)CC1*] |
| 2′OC16-U | 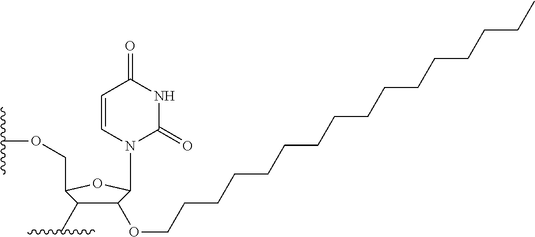 [SMILES: *OCC1OC(n2ccc(=O)[nH]c2=O)C(OCCCCCCCCCCCCCCCC)C1*] |
| Beta-L-DNA | 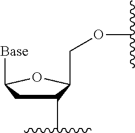 |
| LNA | 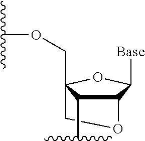 |
| 2′OMe | 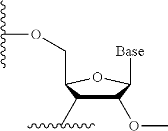 |
| 2′MOE | 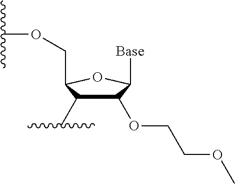 |
| 2′F | 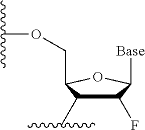 |
| Morpholino | 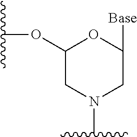 |

Base = A/U/G/C/T/5MeC/5MeU/M6A/Ψ

In a preferred embodiment, R denotes a synthetic 3′ terminus (ii) according to the invention selected from the group of formula L001 to L012:

| ID | Formula |
|---|---|
| L001 | 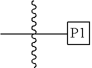 |
| L002 | 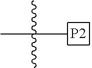 |
| L003 | 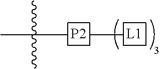 |
| L004 | 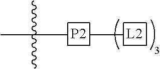 |
| L005 | 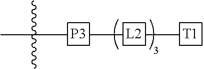 |
| L006 | 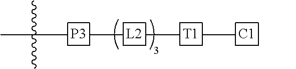 |
| L007 | 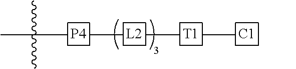 |
| L008 | 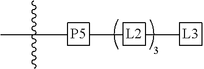 |
| L009 | 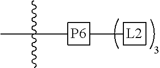 |
| L010 | 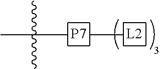 |
| L011 | 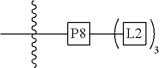 |
| L012 | 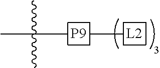 |

wherein
P1 denotes DNA polynucleotide of sequence AAAA,
P2 denotes a polynucleotide according to SEQ ID NO 1
P3 denotes a polynucleotide according to SEQ ID NO 2, wherein nucleobase at position 12 is biotinylated ([Bio-dT]),
P4 denotes a polynucleotide according to SEQ ID NO 3, wherein nucleobase at position 32 is biotinylated,
P5 denotes a polynucleotide according to SEQ ID NO 4, wherein nucleobases at position 21 and 22 are linked to the previous nucleobase by a phosphorothioate bond,
P6 denotes a polynucleotide according to SEQ ID NO 5, wherein nucleobase at position 15 is biotinylated ([Bio-dT]) and nucleobases at position 25 and 26 are linked to the previous nucleobase by a phosphorothioate bond,
P7 denotes a polynucleotide according to SEQ ID NO 6
P8 denotes a polynucleotide according to SEQ ID NO 7, wherein nucleobase at position 4 is biotinylated ([Bio-dT]) and nucleobases at position 25 and 26 are linked to the previous nucleobase by a phosphorothioate bond,
P9 denotes a polynucleotide according to SEQ ID NO 8 wherein nucleobases at position 24 and 25 are linked to the previous nucleobase by a phosphorothioate bond,
T1 denotes a DNA triplet of sequence ACC,
C1 denotes Puromycin,
L1 denotes L-dA,
L2 denotes Sp9,
L3 denotes C3 and
the 3′ end of said polynucleotide M is linked to the 5′ end of said synthetic 3′ terminus.

### INDUSTRIAL APPLICATION

A further aspect of the present invention relates to a method for the generation of the artificial polynucleotide molecule of the present invention comprising or consisting of the following steps:
(i) providing a polynucleotide M (i) and a synthetic 3′ terminus (ii) according to the invention,
(ii) linking said polynucleotide M (i) to the synthetic 3′ terminus (ii), wherein the 3′ end of the polynucleotide M is linked to the synthetic 3′ terminus (ii).

While conventional therapeutical mRNAs such as used in mRNA vaccines are produced by IVT in combination with various enzymatic steps such as polyadenylation, the relatively short artificial polynucleotide molecules according to the invention may be assembled by chemical synthesis and/or using building blocks of preassembled molecules.

The artificial polynucleotide molecule according to the invention may be obtained using known methods in the art. For instance, the polynucleotide M (i) according to the invention may be fully or partially in vitro transcribed. Introduction of a 5′Cap may be facilitated using appropriate capping enzymes as known by the skilled person. Additionally, chemical synthesis of the polynucleotide M (i) may be feasible due to the relatively shorter sequence compared to conventional mRNAs, which comprise a 3′UTR and poly(A)-tail adding to the overall length of the polynucleotide.

Modified natural, e.g., N1-methylpseudouridine, and artificial nucleotides can be incorporated into the polynucleotide M (i) of the invention to further improve stability, enhance translation, and/or reduce immunogenicity of the artificial polynucleotide molecule according to the invention. Surprisingly, the incorporation of at least one chemical modification in the polynucleotide M (i) according to the invention has been shown to further increase the half-life of the artificial polynucleotide molecule according to the invention.

In particular, the combination of multiple different chemical modifications has proven to increase stability further. The unspecific incorporation of chemical modifications into mRNA by IVT does not provide the necessary structural features critical to ensure stability. However, the fully or partially chemical synthesis of the artificial polynucleotide molecule of the invention allows the incorporation of chemical modifications at precise nucleotide positions within the polynucleotide M (i) sequence and, additionally, allows the incorporation of multiple different chemical modifications. Therefore, in one embodiment, the polynucleotide M (i) according to the present invention comprises at least one chemically modified nucleotide. In a preferred embodiment, the polynucleotide M (i) comprises modifications of the ribose 2′ hydroxyl on the RNA backbone selected from the list of 2′OMe nucleotides, 2′-deoxy-2′-fluoro (2′F) nucleotides, 2′-deoxy nucleotides, 2′-O-(2-methoxyethyl) (MOE) nucleotides, locked nucleic acid (LNA) nucleotides, and mixtures thereof.

In a preferred embodiment said at least one modified nucleotide is selected from the group of pseudo-uridine, N1-methyl-pseudo-uridine, phosphotioate links, 2′-OMe, 2′-H, 2′-F, or inverted nucleotides.

The synthetic 3′ terminus (ii) according to the invention may be partially or entirely chemically synthesized. In a preferred embodiment the synthetic 3′ terminus (ii) is obtainable or obtained entirely by chemical synthesis.

These synthesis approaches allow the introduction of chemically modified nucleotides and/or building blocks such as building blocks L and C according to the invention at precisely selected positions of the synthetic 3′ terminus using established and efficient synthesis methods, which enables industrial scalability, increases yield, and reduces costs.

Linking of the polynucleotide M (i) and synthetic 3′ terminus (ii) according to the invention may be achieved by chemically or enzymatically linking the 3′ end of the polynucleotide M (i) to the 5′ end of the synthetic 3′ terminus (ii) by methods known in the art such as the use of ligases.

### Chemical Synthesis of Synthetic 3′ Terminus

The chemical synthesis of the synthetic 3′ terminus according to the invention may be performed using approaches known in the art. For instance, the linking of nucleotides comprising the polynucleotide P of the synthetic 3′ terminus may be performed using methods that are well-known in the art for synthesis of ribonucleic (or deoxyribonucleic) oligonucleotides. Such synthesis is, among others, described in Beaucage and Iyer, Tetrahedron 1992; 48:2223-2311; Beaucage and Iyer, Tetrahedron 1993; 49: 6123-6194 and Caruthers, et. al., Methods Enzymol. 1987; 154: 287-313; the synthesis of thioates is, among others, described in Eckstein, Annu. Rev. Biochem. 1985; 54: 367-402, the synthesis of RNA molecules is described in Sproat, in Humana Press 2005 edited by Herdewijn P.; Kap. 2: 17-31 and respective downstream processes are, among others, described in Pingoud et. al., in IRL Press 1989 edited by Oliver R. W. A.; Kap. 7:183-208.

Other synthetic procedures are known in the art e.g. the procedures as described in Usman et al., 1987, J. Am. Chem. Soc., 109, 7845; Scaringe et al., 1990, NAR., 18, 5433; Wincott et al., 1995, NAR. 23, 2677-2684; and Wincott et al., 1997, Methods Mol. Bio., 74, 59, and these procedures may make use of common nucleic acid protecting and coupling groups, such as dimethoxytrityl at the 5′-end, and phosphoramidites at the 3′-end. The modified (e.g. 2′-O-methylated) nucleotides, unmodified nucleotides, linkers (e.g. Sp9), and conjugates (e.g. Puromycin or GalNAc) are incorporated as desired. It is noted that a commercially available machines can be used for synthesis. Modified nucleotides and building blocks L and C described herein are well known in the art as moieties in polynucleotides. Accordingly, methods for sequential incorporation of said building blocks and modified nucleotides at the 5′ end, 3′ end or between selected nucleotides of a polynucleotide as indicated are well known. For instance, building blocks L such as SpC3, Sp9, SpC12, Sp18, Spermine can be introduced using commercially available building blocks Spacer Phosphoramidite C3 (CAS: 110894-23-0), Spacer Phosphoramidite 9 (CAS: 146668-73-7), Spacer C12 CE Phosphoramidite (CAS: 158665-27-1), Spacer Phosphoramidite 18 (CAS: 125607-09-2), Spermine Phosphoramidite (CAS: 1969276-82-1) respectively.

### Artificial Polynucleotide Molecule Comprising a Protective Group

A, G, C, T/U Phosphoramidites, representing the fundamental monomers for the DNA (A, G, C, T) or RNA (A, G, C, U) bases. A diverse array of phosphoramidite monomers is employed in the solid-phase synthesis of oligonucleotides, incorporating various 2′ protecting groups as recognized in the field. Each monomer may possess a phosphoramidite group protecting at 3′, while the 5′-hydroxyl is equipped with either DMTr, Lev, or Fmoc protecting groups. In some embodiments the artificial polynucleotide molecule comprises a protective group including but not limited to Biotin-dT, 2′OC16-U, abasic site.

In specific embodiments, the building blocks or sub-building blocks described in this invention are chosen from the compounds listed in table 3, encompassing formulas A to L.

TABLE 3

| ID | Name | Formula |
|---|---|---|
| A | dT-CE Phosphoramidite (98796- 51-1) | 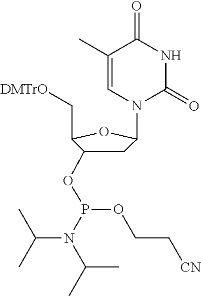 |
| B | dA-CE Phosphoramidite (98796-53-3) | 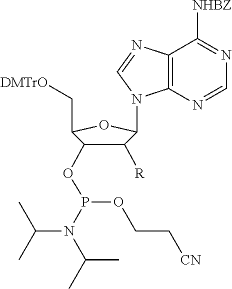 |
| C | dC-CE Phosphoramidite (102212-98-6) | 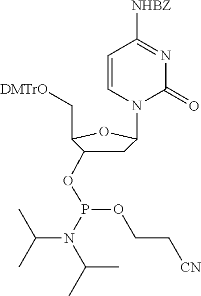 |
| D | dG-CE Phosphoramidite (93183-15-4) | 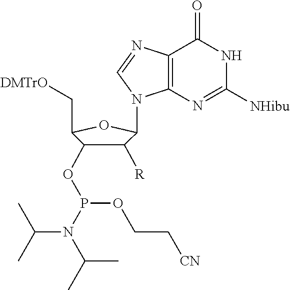 |
| E | 5-FMOC-dT 3′CE Phosphoramidite | 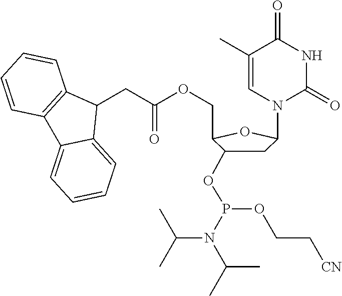 [SMILES: Cc1cn(C2CC(OP(OCCC#N)N(C(C)C)C(C)C)C(COC(=O)CC3c4ccccc4-c4ccccc43)O2)c(=O)[nH]c1=O] |
| F | 5′-FMOC-dG(n-ibu) 3′CE Phosphoramidite | 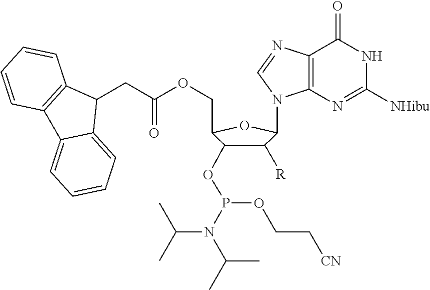 |
| G | 5′-FMOC-dC(n-Bz) 3′CE Phosphoramidite | 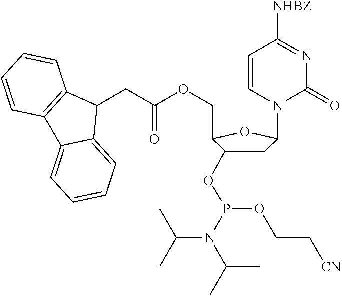 |
| H | 5′-FMOC-dA(n-Bz) 3′CE Phosphoramidite | 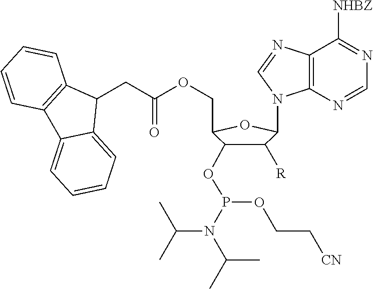 |
| I | 5′-Levulinyl-dT 3′CE Phosphoramidite | 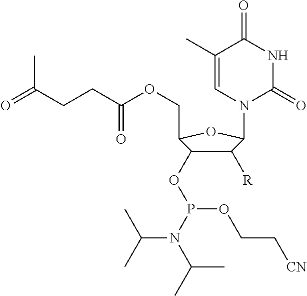 |
| J | 5′-Levulinyl-dG(n-ibu) 3′CE Phosphoramidite | 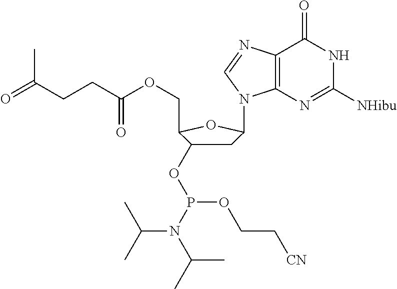 |
| K | 5′-Levulinyl-dC(n-Bz) 3′CE Phosphoramidite | 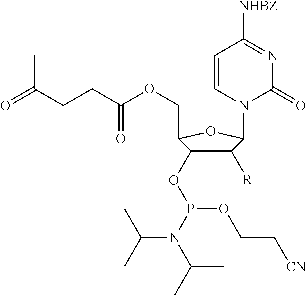 |
| L | 5′-Levulinyl-dA(n-Bz) 3′CE Phosphoramidite | 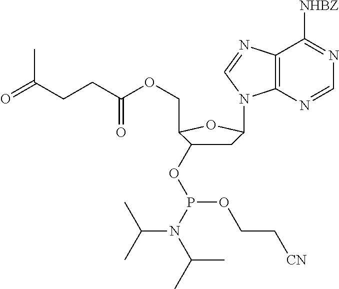 |

In some embodiments the polynucleotide M (i) according to the invention comprises, instead of a 5′Cap, at its 5′ end a removable protecting group. In a preferred embodiment, said protective group is selected from the group of levulinyl, [bis-(4-methoxyphenyl)phenylmethyl](DMTr), and fluorenylmethoxycarbonyl (FMOC), most preferably said protective group is DMTr.

### Linkers

The linkers groups employed in the present invention may exhibit diverse structures, substituents, and substitution patterns. They can be modified with nitrogen, oxygen, and/or sulfur-containing groups, either pendant from or integral to the backbone of the linker group. Examples of such groups include polyethers, polyacids (such as polyacrylic acid and polylactic acid), polyols (e.g., glycerol), polyamines (e.g., spermine, spermidine), and molecules containing multiple nitrogen, oxygen, and/or sulfur moieties (e.g., 1,3-diamino-2-propanol, taurine). Relevant literature, such as Sandier et al.'s “Organic Functional Group Preparations, 2nd Ed., Academic Press, Inc., San Diego, 1983,” provides further details. A broad selection of commercially available mono-, di-, and bis-functionalized poly(ethyleneglycol) molecules can be applied effectively in this aspect of the invention. Refer to sources like the 1997-1998 Catalog from Shearwater Polymers, Inc., Huntsville, Alabama, for specific examples. Moreover, individuals skilled in the field can employ various modification strategies readily within their synthetic repertoire. References like Harris' “Rev. Macromol. Chem. Phys., C25 (3): 325-373 (1985)” and Zalipsky et al.'s “Eur. Polym. J., 19 (12): 1177-1183 (1983)” offer valuable insights. Additionally, U.S. Pat. No. 5,122,614 (issued on Jun. 16, 1992, to Zalipsky) and U.S. Pat. No. 5,650,234 (issued to Dolence et al. on Jul. 22, 1997), along with their respective references, detail useful modification strategies. Furthermore, a range of studies have explored the solid phase synthesis of oligonucleotides with phosphorothioate modifications. Morvan (Tet. Lett. 49. 7149-7152, 1990) successfully synthesized a phosphorothioate oligoribonucleotide on solid support, demonstrating its enhanced resistance to enzymatic degradation and its ability to bind to complementary RNA strands. This work was further expanded by Efimov (Nucleosides, Nucleotides, and Nucleic Acids, 26, 1087-1093, 2007), who developed a method for synthesizing natural and modified oligonucleotides, including those with phosphorothioate analogues, using the phosphotriester technique. Kostov (Molecules, 24(10), 1872, 2019) built on these findings by developing a robust solid-phase protocol for synthesizing chimeric oligonucleotides with various modifications, including phosphorothioate linkages. Lastly, Eldrup (Tet. Lett. 36, 6127-6130, 1990)) introduced new dithiophosphorylating reagents for solid phase synthesis, achieving good coupling efficiencies and producing phosphorodithioate oligodeoxythymidines free from phosphorothioate contaminations. These studies collectively demonstrate the feasibility and potential of solid phase synthesis for producing oligonucleotides with phosphorothioate modifications.

### Therapeutic Polynucleotide Construct

A further aspect of the present invention relates to a therapeutic polynucleotide construct comprising or consisting of the artificial polynucleotide molecule according to the invention, wherein said at least one ORF encodes at least one therapeutic polypeptide.

In some embodiments said therapeutical polypeptide is selected from the group of cytokines and immune system proteins such as immunologically active compounds (e.g., interleukins, colony stimulating factor (CSF), granulocyte colony stimulating factor (G-CSF), granulocyte-macrophage colony stimulating factor (GM-CSF), erythropoietin, tumor necrosis factor (TNF), interferons, integrins, addressins, seletins, homing receptors, T cell receptors, immunoglobulins, soluble major histocompatibility complex antigens, immunologically active antigens such as bacterial, parasitic, or viral antigens, allergens, autoantigens, antibodies), hormones (insulin, thyroid hormone, catecholamines, gonadotrophines, trophic hormones, prolactin, oxytocin, dopamine, bovine somatotropin, leptins and the like), growth hormones (e.g., human grown hormone), growth factors (e.g., epidermal growth factor, nerve growth factor, insulin-like growth factor and the like), growth factor receptors, enzymes (tissue plasminogen activator, streptokinase, cholesterol biosynthetic or degradative, steriodogenic enzymes, kinases, phosphodiesterases, methylases, de-methylases, dehydrogenases, cellulases, proteases, lipases, phospholipases, aromatases, cytochromes, adenylate or guanylaste cyclases, neuramidases and the like), receptors (steroid hormone receptors, peptide receptors), binding proteins (growth hormone or growth factor binding proteins and the like), transcription and translation factors, tumor growth suppressing proteins (e.g., proteins which inhibit angiogenesis), structural proteins (such as collagen, fibroin, fibrinogen, elastin, tubulin, actin, and myosin), blood proteins (thrombin, serum albumin, Factor VII, Factor VIII, insulin, Factor IX, Factor X, tissue plasminogen activator, protein C, von Willebrand factor, antithrombin Ill, glucocerebrosidase, erythropoietin granulocyte colony stimulating factor (GCSF) or modified Factor VIII, anticoagulants and the like. In a preferred embodiment, said therapeutical polypeptide is selected from the group of GLP-1 and insulin.

### Pharmaceutical Compositions

A further aspect of the present invention relates to a pharmaceutical composition comprising or consisting of the therapeutic polynucleotide construct of the invention. In some embodiments said pharmaceutical composition comprises one or more pharmaceutically acceptable diluents and/or excipients and/or one or more adjuvants. Non limiting examples of excipients for a pharmaceutical composition include antioxidants, suspending agents, dispersing agents, preservatives, buffering agents, tonicity agents, and surfactants.

### Delivery Reagents

In some embodiments said pharmaceutical composition further comprises at least one delivery reagent. Delivery reagents useful for the protection and transport of therapeutic nucleic acid constructs in subjects in need thereof may comprise carriers known in the art such as, but not limited to, liposomes, lipoplexes, copolymers, such as PLGA, and lipid nanoparticles (LNPs).

Lipid-based delivery reagents have been increasingly recognized as one of the most promising delivery systems for RNA due to their biocompatibility and their ease of large-scale production. Cationic lipids have been widely studied as synthetic materials for delivery of RNA. After mixing, nucleic acids are condensed by cationic lipids to form lipid/nucleic acid complexes known as lipoplexes. These lipid complexes can protect genetic material from the action of nucleases and to deliver it into cells by interacting with the negatively charged cell membrane. Lipoplexes can be prepared by directly mixing positively charged lipids at physiological pH with negatively charged nucleic acids. In some embodiments, the at least one deliver reagent of the pharmaceutical composition according to the invention is one or more lipids selected from cationic lipids, ionizable lipids, anionic lipids, sterols, pegylated lipids, and any combination of the foregoing. In some embodiments, the pharmaceutical composition containing a translatable compound comprises a cationic lipid, a phospholipid, cholesterol, and a pegylated lipid. In certain embodiments, a pharmaceutical composition can be substantially free of liposomes.

In further embodiments, the at least one deliver reagent of the pharmaceutical composition according to the invention is a lipid nanoparticle (LNP). In some embodiments said LNP comprises at least one regent selected from the group of cationic or ionizable lipids, an aggregation reducing agent (such as polyethylene glycol (PEG) lipid or PEG-modified lipid), a non-cationic lipid (such as a neutral lipid), and a sterol.

Precise delivery of pharmaceutical compositions according to the invention is necessary to ensure delivery to host cells useful for the translation of the therapeutic polypeptide encoded. Deliver reagents according to the present invention and the salts, solvates and physiologically functional derivatives thereof, therefore, may include monoclonal antibodies, nucleic acids or nanoparticles as individual carriers to which the synthetic mRNA constructs are coupled or enclosed allowing the delivery of the pharmaceutical composition to desired host cells. The delivery reagent can guide the delivery of the synthetic mRNA construct by engaging with known biological structures of host cells such as cell-specific receptors and surface markers.

In addition, if desired or necessary, suitable binders, lubricants and disintegrants as well as dyes can likewise be incorporated into the mixture. Suitable binders include starch, gelatin, natural sugars, such as, for example, glucose or beta-lactose, sweeteners made from maize, natural and synthetic rubber, such as, for example, acacia, tragacanth or sodium alginate, carboxymethylcellulose, polyethylene glycol, waxes, and the like. The lubricants used in these dosage forms include sodium oleate, sodium stearate, magnesium stearate, sodium benzoate, sodium acetate, sodium chloride and the like. The disintegrants include, without being restricted thereto, starch, methylcellulose, agar, bentonite, xanthan gum and the like. The tablets are formulated by, for example, preparing a powder mixture, granulating or dry-pressing the mixture, adding a lubricant and a disintegrant and pressing the entire mixture to give tablets. A powder mixture is prepared by mixing the compound comminuted in a suitable manner with a diluent or a base, as described above, and optionally with a binder, such as, for example, carboxymethylcellulose, an alginate, gelatin or polyvinylpyrrolidone, a dissolution retardant, such as, for example, paraffin, an absorption accelerator, such as, for example, a quaternary salt, and/or an absorbent, such as, for example, bentonite, kaolin or dicalcium phosphate. The powder mixture can be granulated by wetting it with a binder, such as, for example, syrup, starch paste, acacia mucilage or solutions of cellulose or polymer materials and pressing it through a sieve. As an alternative to granulation, the powder mixture can be run through a tableting machine, giving lumps of non-uniform shape, which are broken up to form granules. The granules can be lubricated by addition of stearic acid, a stearate salt, talc or mineral oil in order to prevent sticking to the tablet casting molds. The lubricated mixture is then pressed to give tablets. The compounds according to the invention can also be combined with a free-flowing inert excipient and then pressed directly to give tablets without carrying out the granulation or dry-pressing steps. A transparent or opaque protective layer consisting of a shellac sealing layer, a layer of sugar or polymer material and a gloss layer of wax may be present. Dyes can be added to these coatings in order to be able to differentiate between different dosage units.

Oral liquids, such as, for example, solution, syrups, and elixirs, can be prepared in the form of dosage units so that a given quantity comprises a pre-specified amount of the compound. Syrups can be prepared by dissolving the compound in an aqueous solution with a suitable flavor, while elixirs are prepared using a non-toxic alcoholic vehicle. Suspensions can be formulated by dispersion of the compound in a non-toxic vehicle. Solubilizers and emulsifiers, such as, for example, ethoxylated isostearyl alcohols and polyoxyethylene sorbitol ethers, preservatives, flavor additives, such as, for example, peppermint oil or natural sweeteners or saccharin, or other artificial sweeteners and the like, can likewise be added. The dosage unit formulations for oral administration can, if desired, be encapsulated in microcapsules. The formulation can also be prepared in such a way that the release is extended or retarded, such as, for example, by coating or embedding of particulate material in polymers, wax and the like.

All acid and base salts of the compounds described herein are intended to be included within the scope of this invention. A compound may exist in an unsolvated or solvated form, including hydrated forms. In general, the solvated forms, with pharmaceutically acceptable solvents such as water, ethanol, and the like, are equivalent to the unsolvated forms for the purposes of this disclosure. Compounds, salts, and solvates thereof, may exist in a tautomeric form, for example, as an amide or imino ether. All tautomeric forms are included in this invention.

### Administration

Administering a therapeutically effective amount of the pharmaceutical composition according to the invention can be achieved by any means known in the art such as intravenously, subcutaneously, intraperitoneally, orally, or through inhalation.

A pharmaceutical composition according to the invention can be capable of local or systemic administration. In some aspects, a pharmaceutical composition can be capable of any modality of administration. In certain aspects, the administration can be by any route, including intravenous, subcutaneous, pulmonary, intramuscular, intraperitoneal, dermal, oral, inhalation or nasal administration.

In some embodiment according to the present invention, the pharmaceutical composition can be administered alone or in combination with an additional co-agent such as pharmaceutically active compounds or, useful in the treatment and/or prevention of diabetes mellitus and/or obesity.

In some embodiment the co-agent is administered in a prophylaxis-effective amount or a treatment-effective amount.

Pharmaceutical compositions can be adapted for administration via any desired suitable method, for example by oral (including buccal or sublingual), rectal, nasal, topical (including buccal, sublingual or transdermal), vaginal or parenteral (including subcutaneous, intramuscular, intravenous or intradermal) methods. Such formulations can be prepared using all processes known in the pharmaceutical art by, for example, combining the active ingredient with the excipient(s) or adjuvant(s).

In some embodiments, the administration according to the method of the present invention takes place oral, including buccal or sublingual, rectal, nasal, topical, including buccal, sublingual or transdermal, vaginal or parenteral, including subcutaneous, intramuscular, intravenous or intradermal.

Pharmaceutical compositions adapted for oral administration can be administered as separate units, such as, for example, capsules or tablets; powders or granules; solutions or suspensions in aqueous or non-aqueous liquids; edible foams or foam foods; or oil-in-water liquid emulsions or water-in-oil liquid emulsions.

Thus, for example, in the case of oral administration in the form of a tablet or capsule, the active-ingredient component can be combined with an oral, non-toxic and pharmaceutically acceptable inert excipient, such as, for example, ethanol, glycerol, water and the like. Powders are prepared by comminuting the compound to a suitable fine size and mixing it with a pharmaceutical excipient comminuted in a similar manner, such as, for example, an edible carbohydrate, such as, for example, starch or mannitol. A flavor, preservative, dispersant and dye may likewise be present.

Capsules are produced by preparing a powder mixture as described above and filling shaped gelatin shells therewith. Glidants and lubricants, such as, for example, highly disperse silicic acid, talc, magnesium stearate, calcium stearate or polyethylene glycol in solid form, can be added to the powder mixture before the filling operation. A disintegrant or solubilize, such as, for example, agar-agar, calcium carbonate or sodium carbonate, may likewise be added in order to improve the availability of the medicament after the capsule has been taken.

Pharmaceutical compositions adapted for transdermal administration can be administered as independent plasters for extended, close contact with the epidermis of the recipient. Thus, for example, the active ingredient can be delivered from the plaster by iontophoresis, as described in general terms in Pharmaceutical Research, 3(6), 318 (1986).

Pharmaceutical compounds adapted for topical administration can be formulated as ointments, creams, suspensions, lotions, powders, solutions, pastes, gels, sprays, aerosols or oils.

For the treatment of the eye or other external tissue, for example mouth and skin, the formulations are preferably applied as topical ointment or cream. In the case of formulation to give an ointment, the active ingredient can be employed either with a paraffinic or a water-miscible cream base. Alternatively, the active ingredient can be formulated to give a cream with an oil-in-water cream base or a water-in-oil base.

Pharmaceutical compositions adapted for topical application to the eye include eye drops, in which the active ingredient is dissolved or suspended in a suitable carrier, in particular an aqueous solvent.

Pharmaceutical compositions adapted for topical application in the mouth encompass lozenges, pastilles and mouthwashes.

Pharmaceutical compositions adapted for rectal administration can be administered in the form of suppositories or enemas.

Pharmaceutical compositions adapted for nasal administration in which the carrier substance is a solid comprise a coarse powder having a particle size, for example, in the range 20-500 microns, which is administered in the manner in which snuff is taken, i.e. by rapid inhalation via the nasal passages from a container containing the powder held close to the nose. Suitable formulations for administration as nasal spray or nose drops with a liquid as carrier substance encompass active-ingredient solutions in water or oil. Pharmaceutical formulations adapted for administration by inhalation encompass finely particulate dusts or mists, which can be generated by various types of pressurized dispensers with aerosols, nebulizers or insufflators.

Pharmaceutical compositions adapted for vaginal administration can be administered as pessaries, tampons, creams, gels, pastes, foams or spray formulations.

Pharmaceutical compositions adapted for parenteral administration include aqueous and non-aqueous sterile injection solutions comprising antioxidants, buffers, bacteriostatics and solutes, by means of which the formulation is rendered isotonic with the blood of the recipient to be treated; and aqueous and non-aqueous sterile suspensions, which may comprise suspension media and thickeners. The formulations can be administered in single-dose or multidose containers, for example sealed ampoules and vials, and stored in freeze-dried (lyophilized) state, so that only the addition of the sterile carrier liquid, for example water for injection purposes, immediately before use is necessary. Injection solutions and suspensions prepared in accordance with the recipe can be prepared from sterile powders, granules and tablets.

It goes without saying that, in addition to the above particularly mentioned constituents, the compositions may also comprise other agents usual in the art with respect to the particular type of formulation; thus, for example, formulations which are suitable for oral administration may comprise flavors.

### Medical Use

The artificial polynucleotide molecules, therapeutic polynucleotide constructs, and pharmaceutical compositions according to the present invention are ideal tools to produce therapeutic polypeptides within host cells able to exert biological functions in target cells, tissues, and environments and can be used alone or further modified to form pharmaceutical compositions useful for the treatment of diseases. Therefore, the present invention further relates to the artificial polynucleotide molecules, therapeutic polynucleotide constructs, and pharmaceutical compositions according to the present invention for use as a medicament.

The disclosure further provides a method to protect a subject from an indication selected from the group consisting of infectious disease, diabetes and obesity. Said method comprises:
a) providing a pharmaceutical composition according to the invention and
b) administering to a host cell of a subject said pharmaceutical composition,
wherein administration of said composition protects the subject from an indication selected from the group consisting of infectious disease, diabetes and obesity.

In one embodiment said method protects a subject from Type II diabetes. In another embodiment said method protects a subject from Type I diabetes. In another embodiment said method protects a subject from gestational diabetes. In another embodiment said method protects a subject from maturity onset diabetes of the young (MODY). In another embodiment said method protects a subject from obesity. In another embodiment, the said method protects a subject from a monogenic form of obesity or diabetes (e.g., Type 2 diabetes). In another embodiment said method protects a subject from a polygenic form of obesity or diabetes (e.g., Type 2 diabetes).

As used herein, a subject is any animal that is susceptible disease. Subjects include humans and other mammals, such as cats, dogs, horses, other companion animals, other zoo animals, lab animals (e.g., mice, rats), and livestock.

A host cell according to the present invention refers to any cell capable of producing mRNA-encoded polypeptides and optionally secreting said polypeptide either to an epithelial surface or directly to the bloodstream.

In some embodiments, said host cell is selected from the list of exocrine cells and endocrine cells. In some embodiments said host cell is selected from the group of Brunner's gland cell in duodenum, Insulated goblet cell of respiratory and digestive tracts, Foveolar cell, Chief cell, Parietal cell, Pancreatic acinar cell, Paneth cell of small intestine, Type II pneumocyte of lung, Club cell of lung, Type I pneumocyte, Gall bladder epithelial cell, Centroacinar cell, Intercalated duct cell, Intestinal brush border cell, K cell, L cell, I cell, G cell, Enterochromaffin cell, Enterochromaffin-like cell, N cell, S cell, D cell, Mo cell, Thyroid gland cells, Thyroid epithelial cell, Parafollicular cell, Parathyroid gland cells, Parathyroid chief cell, Oxyphil cell, Pancreatic islets, Alpha cell, Beta cell, Delta cell, Epsilon cell, PP cell, Salivary gland mucous cell, Salivary gland serous cell, Von Ebner's gland cell in tongue, Mammary gland cell, Lacrimal gland cell, Ceruminous gland cell in ear, Eccrine sweat gland dark cell, Eccrine sweat gland clear cell, Apocrine sweat gland cell, Gland of Moll cell in eyelid, Sebaceous gland cell, Bowman's gland cell in nose, Hormone-secreting cells, Anterior/Intermediate pituitary cells, Corticotropes, Gonadotropes, Lactotropes, Melanotropes, Somatotropes, Thyrotropes, Magnocellular neurosecretory cells, Parvocellular neurosecretory cells, Chromaffin cells, Epithelial cells, Keratinocyte, Epidermal basal cell, Melanocyte, Trichocyte, Medullary hair shaft cell, Cortical hair shaft cell, Cuticular hair shaft cell, Huxley's layer hair root sheath cell, Henle's layer hair root sheath cell, Outer root sheath hair cell, Surface epithelial cell of cornea, tongue, mouth, nasal cavity, distal anal canal, distal urethra, and distal vagina, basal cell (stem cell) of cornea, tongue, mouth, nasal cavity, distal anal canal, distal urethra, and distal vagina, Intercalated duct cell, Striated duct cell, Lactiferous duct cell, Ameloblast, Oral cells, Odontoblast, Cementoblast,

Salivary glands are recognized as a useful depot organ in gene therapy, having several important features of other endocrine glands, such as high protein production and ability to secrete proteins into the bloodstream (see, for example, Voutetakis et al., 2005, J Endocrinol 185, 363-372). It has been previously reported that salivary glands are able to produce pharmacological levels of growth hormone and parathyroid hormone following transduction with recombinant viral vectors (see, for example, He et al., 1998, Gene Ther 5, 537-541; Adriaansen et al., 2011, Hum Gene Ther 22, 84-92). Therefore, in a preferred embodiment, the host cell according to the method of the invention is a salivary gland cell.

The invention encompasses the administration of therapeutic polynucleotide constructs according to the invention or of a pharmaceutical composition thereof, wherein said construct or the pharmaceutical composition thereof is administered to an individual prior to, simultaneously or sequentially with other therapeutic regimens or co-agents useful in the treatment of diabetes and/or obesity, in an effective amount. Compounds according to the present invention or the pharmaceutical formulations thereof that are administered simultaneously with said co-agents can be administered in the same or different composition(s) and by the same or different route(s) of administration.

### Dosage

A therapeutically effective amount of therapeutic polynucleotide construct according to the present invention depends on several factors, including, for example, the age and weight of the subjects such as animals and humans, the precise condition that requires treatment, and its severity, the nature of the formulation and the method of administration, and is ultimately determined by the treating doctor or vet. However, a therapeutically effective dose of an active agent, e.g., a therapeutic polynucleotide construct or pharmaceutical composition according to the invention, in vivo can be a dose of about 0.001 to about 500 mg/kg body weight. For instance, the therapeutically effective dose may be about 0.001-0.01 mg/kg body weight, or 0.01-0.1 mg/kg, or 0.1-1 mg/kg, or 1-10 mg/kg, or 10-100 mg/kg. In some embodiments, a therapeutic polynucleotide construct or pharmaceutical composition according to the invention can be provided at a dose ranging from about 0.1 to about 10 mg/kg body weight, e.g., from about 0.5 to about 5 mg/kg, from about 1 to about 4.5 mg/kg, or from about 2 to about 4 mg/kg.

A therapeutically effective dose of an active agent, e.g., a therapeutic polynucleotide construct or pharmaceutical composition according to the invention, in vivo can be a dose of at least about 0.001 mg/kg body weight, or at least about 0.01 mg/kg, or at least about 0.1 mg/kg, or at least about 1 mg/kg, or at least about 2 mg/kg, or at least about 3 mg/kg, or at least about 4 mg/kg, or at least about 5 mg/kg, at least about 10 mg/kg, at least about 20 mg/kg, at least about 50 mg/kg, or more. In some embodiments, a synthetic mRNA construct or pharmaceutical composition according to the invention can be provided at a dose of about 0.1 mg/kg, about 0.5 mg/kg, about 1 mg/kg, about 1.5 mg/kg, about 2 mg/kg, about 2.5 mg/kg, about 3 mg/kg, about 3.5 mg/kg, about 4 mg/kg, about 5 mg/kg, or about 6, 7, 8, 9, 10, 15, 20, 25, 50, 75, or 100 mg/kg.

Further preferred embodiments listed above also apply to the method according to the present invention.

A further aspect of the present invention relates to a synthetic 3′ terminus compound according to formula S0:

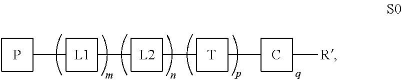

wherein
P denotes a polynucleotide P of the synthetic 3′terminus (ii) according to the invention,
L1 denotes a first building block L according to the invention,
L2 denotes a second building block L according to the invention,
T denotes a DNA triplet,
C denotes a building block C according to the invention,
m, n denote independently from each other an integer between 0 and 10,
p, q denotes independently from each 0 or 1,
R′ denote H, OH, canonical or, non-canonical nucleotide either individually or in conjunction with a delivery conjugate including but not limited to 5′GalNAc, CholTeG, Biotine
L1 and L2 are independently selected from the group of SpC3, Sp9, SpC12, Sp18, Spermine, 1-Ethynyl-dSpacer, CholTEG, L-dA, Bio-TEG linkers, and GalNAc,
L1 and L2 are different from each other and,
C is selected from the group of Puromycin, biotin-dT, 2′OC16-U, Beta-L-DNA, LNA, 2′Ome, 2′MOE, 2′F, Morpholino.

In a preferred embodiment R′ is H or OH, more preferably OH.

The polynucleotide P of the Formula SO is depicted in 5′ to 3′ direction, wherein the 5′ end of the first building block is linked to the 3′ end of the polynucleotide P. Each building block is linked to the previous building block according to the polynucleotide synthesis nomenclature from 5′ to 3′ by e.g., phosphodiester bonds (unless otherwise stated) as known in the arts.

In a preferred embodiment, P is a DNA polynucleotide of length between 4 and 50 nt. Preferably, P is selected from the group of DNA polynucleotide of sequence AAAA and polynucleotide according to SEQ ID NOs 1-8.

In a preferred embodiment, Li and L2 are independently selected from the group of SpC3 and Sp9. Preferably, m is an integer of between 1 and 3, n is 0 and Li is Sp9. In a preferred embodiment, q is 1 and C is Puromycin.

In a preferred embodiment the synthetic 3′ terminus compound is a compound according to formula ST1 to ST10:

| ID | Formula |
|---|---|
| ST1 | 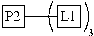 |
| ST2 | 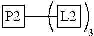 |
| ST3 | 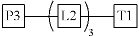 |
| ST4 | 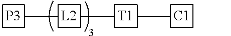 |
| ST5 | 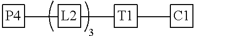 |
| ST6 | 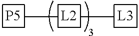 |
| ST7 | 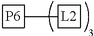 |
| ST8 | 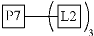 |
| ST9 | 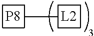 |
| ST10 | 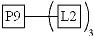 |

wherein
P2 denotes a polynucleotide according to SEQ ID NO 1
P3 denotes a polynucleotide according to SEQ ID NO 2, wherein nucleobase at position 12 is biotinylated ([Bio-dT]),
P4 denotes a polynucleotide according to SEQ ID NO 3, wherein nucleobase at position 32 is biotinylated,
P5 denotes a polynucleotide according to SEQ ID NO 4, wherein nucleobases at position 21 and 22 are linked to the previous nucleobase by a phosphorothioate bond,
P6 denotes a polynucleotide according to SEQ ID NO 5, wherein nucleobase at position 15 is biotinylated ([Bio-dT]) and nucleobases at position 25 and 26 are linked to the previous nucleobase by a phosphorothioate bond,
P7 denotes a polynucleotide according to SEQ ID NO 6
P8 denotes a polynucleotide according to SEQ ID NO 7, wherein nucleobase at position 4 is biotinylated ([Bio-dT]) and nucleobases at position 25 and 26 are linked to the previous nucleobase by a phosphorothioate bond,
P9 denotes a polynucleotide according to SEQ ID NO 8 wherein nucleobases at position 24 and 25 are linked to the previous nucleobase by a phosphorothioate bond,
T1 denotes a DNA triplet of sequence ACC,
C1 denotes Puromycin,
L1 denotes L-dA,
L2 denotes Sp9, and
L3 denotes C3.

The synthetic 3′ terminus compounds according to the present invention, surprisingly, provide compounds useful for the stabilization of an RNA or RNA-like molecule. While RNA in vitro and in vivo are prone to chemical and/or enzymatic degradation, RNA molecules linked at their 3′ terminus with the synthetic 3′ terminus of the invention show significantly increased half-life in vitro and in vivo. In particular, mRNA fragments such as an open reading frame, in combination with a 5′Cap and 5′ UTR can be easily functionalized with the synthetic 3′ terminus compounds according to the invention, wherein the linked synthetic 3′ terminus compounds stabilize the mRNA fragments, and/or exert a stabilizing and/or enhancing function on translation compared to a reference mRNA.

Conveniently, the synthetic 3′ terminus compounds according to the invention can be produced either completely by chemical synthesis or a combination of IVT and chemical synthesis as known in the art, thereby allowing efficient and economically-viable production. Synthetic 3′ terminus compounds according to the invention can be stored in solution or solid phase thereby providing a convenient reagent for the generation of RNA molecules for various functions such as therapeutic mRNA.

In some embodiments the synthetic 3′ terminus according to the present invention further comprises at the 5′ terminus RNA nucleotides. Said further RNA nucleotides may be useful for the linkage of RNA molecules such as mRNAs or mRNA-like molecules that can be functionally linked to the synthetic 3′ terminus. In some embodiments said synthetic 3′ terminus compounds according to the present invention comprise at their 5′ terminus between about 1 nt and about 200 nt, more preferably between about 2 and about 100 nt, more preferably between about 3 and about 50 nt, more preferably between about 5 and about 40 nt, and most preferably about 1 to about 10 nt.

### Kits

The artificial polynucleotide molecule, the synthetic 3′ terminus compounds, the therapeutic polynucleotide construct, and the pharmaceutical composition and ingredients of said pharmaceutical composition may also be manufactured and traded separately of each other. Thus, the invention relates further to a kit or kit of parts comprising the artificial polynucleotide molecule, the synthetic 3′ terminus compound, the therapeutic polynucleotide construct, and/or the pharmaceutical composition according to the invention. Preferably, such kit or kits of parts may, additionally, comprise instructions for use, cells for transfection, an adjuvant, a means for administration of the pharmaceutical composition, a pharmaceutically acceptable carrier and/or a pharmaceutically acceptable solution for dissolution or dilution of the artificial nucleic acid molecule, the vector, the cells, or the pharmaceutical composition.

### BRIEF DESCRIPTION OF THE FIGURES

FIG. 1 shows the effect of fully non-natural synthetic 3′ termini according to the invention on HiBiT activity in A549 cells. A) Design of HiBiT mRNA with synthetic 3′ termini and control constructs. Control RNAs: HiBiT RNA-poly(A): mRNA without 3′UTR or poly(A); HiBiT RNA+poly(A): mRNA with 20A poly(A)-RNA; xRNA v0.1.1+Puromycin (L006): the chimeric construct shown in FIG. 1A, first row; xRNA v0.1.1—Puromycin (L005): the chimeric construct shown in FIG. 1A, second row. B) HiBiT activity (Relative Luminescence Unit, RLU) measured 6 h post-transfection in A549 cells. Mock corresponds to cells transfected with Lipofectamine MessengerMax only. Error bars denote the standard error of the mean (SEM). n=3. Depicted polynucleotide sequences in FIG. 1 A correspond to SEQ ID NO 2 for xRNA v.0.1.1+Puromycin and xRNA v.0.1.1—Puromycin, and SEQ ID NO 9 for HiBit RNA+poly(A).

FIG. 2 shows Long-term HiBiT activity measurements in A549 cells transfected with xRNA constructs. A) xRNA v0.0.1 corresponds to the ligation product of S8_069 to in vitro transcribed and capped (cap1) HiBiT mRNA as in FIG. 4A. xRNA v0.1.1. Respective mRNA controls were generated via in vitro transcription and enzymatic capping (cap1). B) HiBiT activity measurements over a 96 h time course experiments conducted in transfected A549 cells with different xRNA versions and their respective mRNA controls (ctrl). RLU=Relative Luminescence Unit. Error bars represent the standard error of the mean (SEM). n=3. Depicted polynucleotide sequence in FIG. 2 A corresponds to SEQ ID NO 10 for xRNA v.0.0.1 (sequence 1), SEQ ID NO 11 for mRNA control (sequence 2), and SEQ ID NO 12 for xRNA v.0.1.1 (sequence 3), and SEQ ID NO 13 for mRNA control (sequence 4).

FIG. 3 shows the Effect of a non-poly(A) DNA sequence on HiBiT activity. A) Schematic of RNA-DNA chimera with non-poly(A) DNA sequence. B) HiBiT activity measurements 6 h post-transfection of A549 cells transfected with constructs shown in FIG. 2A. C) HiBiT activity measurements in A549 cells transfected with constructs shown in FIG. 2A and measured at various time points up to 96 h post-transfection. RLU=Relative Luminescence Unit. Error bars denote the standard error of the mean (SEM). n=3. Depicted polynucleotide sequence in FIG. 3 A corresponds to SEQ ID NO 9 for mRNA control and SEQ ID NO 14 for xRNA: non-poly(A).

FIG. 4 shows HiBiT activity of various 3′end modified HiBiT RNA. A) Various conventional and synthetic 3′ termini were ligated onto in vitro transcribed and capped (cap1) HiBiT mRNA. In addition, an in vitro transcribed, cap1-mRNA corresponding to S8_070+S8_084 was generated: S8_072. Constructs were transfected in A549 or Hek293T as indicated. B) HiBiT activity measurements of a subset of constructs (as indicated in panel A) 6 h post-transfection in A549 cells. C+D) HiBiT activity measurements of additional constructs (as indicated in panel A) side-by-side with best performing constructs in panel B. Measurements were taken 6 h post-transfection in C) A549 and D) Hek293T. RLU=Relative Luminescence Unit. Error bars denote the standard error of the mean (SEM). n=3.

FIG. 5 shows HiBiT activity of 3′end modified HiBiT RNA 48 h post-transfection in A549 cells. Constructs tested correspond to 58_069 (25dA+3×Sp9), S8_036 (PS+GalNAc) and S8_089 (3×L-DNA). RLU=Relative Luminescence Unit. Error bars denote the standard error of the mean (SEM). n=3.

FIG. 6 shows effects of a synthetic 3′ terminus on functional GLP-1 expression. A) Schematic of tested constructs. Key: *=phosphorothioate bond; iSp9=internal Spacer9; 3Sp9=3′ Spacer9. B) Secretion efficiency of functional GLP-1 produced in transfected A549 cells as measured using a GLP-1 reporter cell system. black=chimeric GLP-1 (V3.1), grey=mRNA control (V2). C) Time course to assess active GLP-1 protein levels in cell lysates of transfected A549 cells as measured by ELISA. black=chimeric GLP-1 (V3.1), grey=mRNA control (V2). Shown are the fold changes in Relative Luminescence Unit (RLU) relative to mock transfected (Lipofectamine only) controls. Error bars represent the standard error of the mean (SEM). n=3 in case of (B) and n=2 in case of (C). Depicted polynucleotide sequence in FIG. 6 A corresponds to SEQ ID NO 8 for V3.1 3′ end.

FIG. 7 shows effects of a synthetic 3′ terminus on total GLP-1 expression. Total secreted GLP-1 protein levels were assessed in transfected A549 cells by ELISA on cell supernatant collected A) 24 h and B) 48 h post-transfection. Error bars represent the standard error of the mean (SEM). n=2.

FIG. 8 shows comparative analysis of chimeric GLP-1 constructs in A549 cells functionalized with indicated synthetic 3′ termini. A) Schematic of constructs tested. Key: *=phosphorothioate bond; iSp9=internal Spacer9; 3Sp9=3′ Spacer9; BiodT=Biotin-dT. B) Time course experiment of transfected A549 cells. Active GLP-1 levels were measured using a GLP-1 reporter cell line system. Shown is the fold change in Relative Luminescence Unit (RLU) relative to mock transfected (Lipofectamine only) controls. Error bars represent the standard error of the mean (SEM). n=9 for 24 h, n=6 for 6 h and n=3 for 72 h. Depicted polynucleotide sequences in FIG. 8 A corresponds to SEQ ID NO 8 for V3.1 3′ end, SEQ ID NO 7 for V3.2.1 (P03) 3′ end, and SEQ ID NO 5 for V3.2.2 (P15) 3′ end.

FIG. 9 shows effect of a synthetic 3′ termini on mRNA functionality in mouse liver. A) Schematic of constructs delivered to mice livers. B) Time course of xRNA detection from fresh mouse liver lysates. Each data point represents liver lysates from 1 animal; 3 animals were tested per time point/group. Depicted polynucleotide sequences in FIG. 9 A corresponds to SEQ ID NO 9 for cap1-mRNA and SEQ ID NO 4 for cap1-xRNA.

FIG. 10 shows long-term effect of a synthetic 3′ terminus on mRNA functionality in mice. Time course of cap1-xRNA levels in fresh mouse liver lysates. See schematic of constructs in FIG. 9 A. Each data point represents liver lysates from 1 animal; 3 animals were tested per time point/group, except at 144 h for which only 2 animals were tested.

FIG. 11 shows serum cytokine levels at tested time points for A) IP-10 and B) IL-6. Sera collected at 6, 12, 24, 48, 72 & 96 hrs post single dose of Cap1-xRNA, Cap1-mRNA and PBS administration were analyzed by commercially available ELISA kits (n=3). Each data point represents individual animal values (pg/ml). Horizontal lines in the graph represent LLOQ of each cytokine.

FIG. 12 shows the percent body weight change post single dose of PBS/CAP1-mRNA/CAP1-xRNA upto 96 hrs. mRNA treatment groups were terminated at 24 hrs and PBS/CAP1-xRNA treatment groups were followed till 96 hrs. Body weights prior to dosing was considered as baseline (denoted as 0 in the graph).

FIG. 13 shows a purely exemplary and non-limiting depiction of structure R0 according to the invention, wherein P is a polynucleotide of length 3, C1 is Sp9, m is 1, n and q are 0, C is Puromycin, and q is 1.

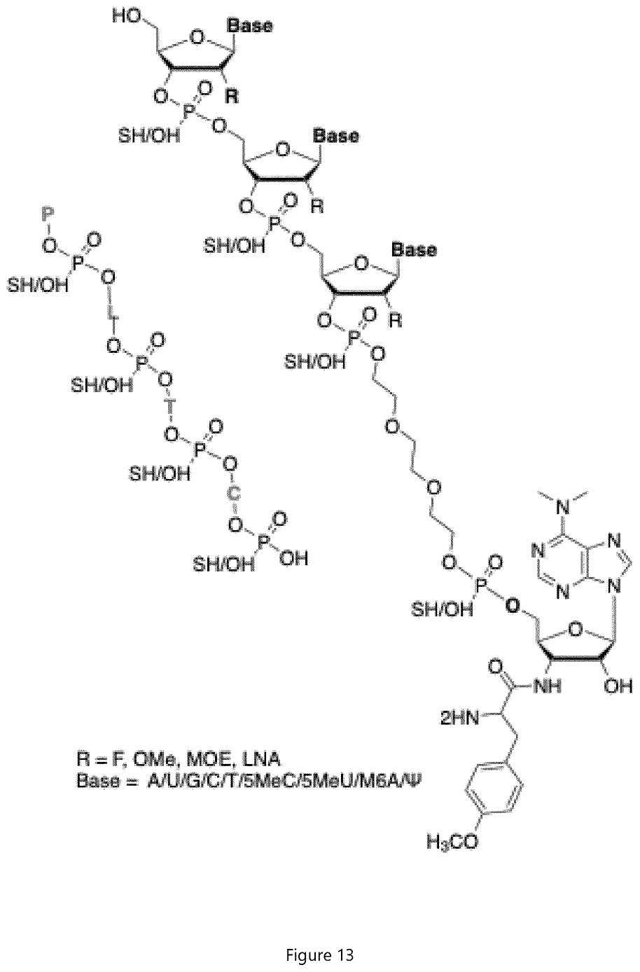

FIG. 14 Effect of CDS modification on stability. A) General sequence of the library constructs comprising the same 5′UTR. Illustrate the designed library of 2,000 sequences encoding the HiBit peptide flanked by Glycine/Serine linkers. B) Experiment results. Each point represents a single mRNA sequence, with black dots indicating sequences without a CDS modification, whereas white does indicate sequences with a CDS modification. Depicted polynucleotide sequences in FIG. 14 A corresponds to SEQ ID NO 15 for forward primer and SEQ ID NO 16 for the general sequence of the library constructs comprising the same 5′UTR, and SEQ ID NO 17 for depicted reverse primer. Amino acid sequence for HiBit according to SEQ ID NO 18, upstream linker according to SEQ ID NO 19, downstream linker according to SEQ ID NO 20.

### EXAMPLES

### Example 1

### A Fully Synthetic 3′ Terminus Enhances Stability and Translational Yield of mRNA In Vitro

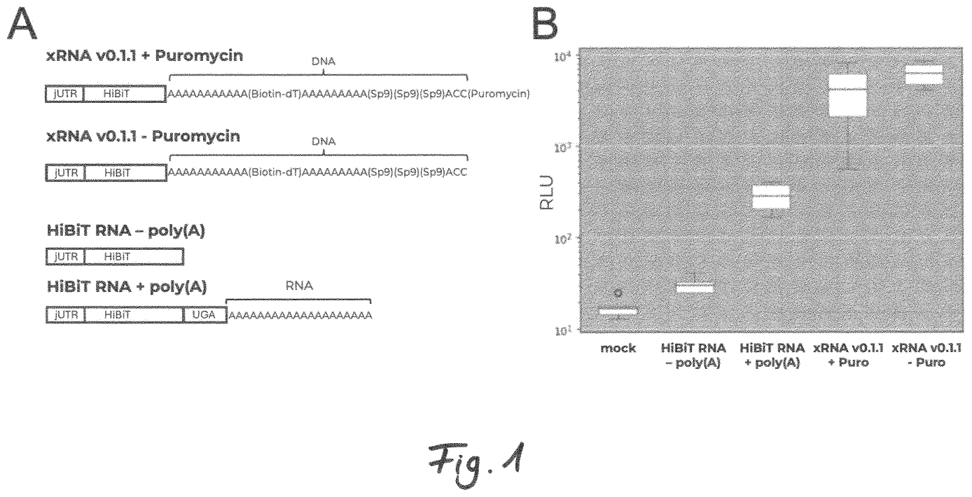

In this experiment we test the feasibility of replacing the canonical 3′-end of an mRNA with an entirely non-natural molecule (synthetic 3′ terminus according to the invention) in order to improve properties such as increase RNA stability and thereby translational yield. We directly compare the luminescence signal generated from cells transfected with non-modified control mRNAs encoding the HiBiT peptide (containing a stop codon, +/−poly(A)-RNA sequence) to non-natural mRNA-DNA chimeras, termed xRNA, also encoding HiBiT (FIG. 1A, xRNA v0.1.1). In the non-natural mRNA-DNA chimeras, the stop codon and 3′UTR were replaced completely with a poly(A)-DNA sequence, as well as additional non-natural elements including biotin-dT, Spacer9 and Puromycin.

### Materials and Methods

The xRNA (+/−Puromycin) were generated by ligating an in vitro transcribed RNA encoding HiBiT to a DNA oligo containing additional modifications, including biotin-dT, Spacer9, with and without Puromycin. The control mRNAs were generated by in vitro transcription. All constructs were capped with Vaccinia capping enzyme and mRNA Cap 2′-O-Methyltransferase (NEB) to produce a Cap-1 structure. Schematics of structures are depicted in FIG. 1 A.

### Complete Oligo Sequences:

HIBIT RNA-poly(A):
SEQ ID NO 21
cap 1-GGGAGAGCCACCAUGGUGAGCGGCUGGCGGCUGUUCAAGA
AGAUUAGCUGA
HIBIT RNA + poly(A):
SEQ ID NO 22
cap 1-GGGAGAGCCACCAUGGUGAGCGGCUGGCGGCUGUUCAAGA
AGAUUAGCUGAAAAAAAAAAAAAAAAAAAAA
xRNA v0.1.1 + Puromycin:
SEQ ID NO 23
cap1-GGGAGAGCCACCAUGGUGAGCGGCUGGCGGCUGUUCAAGAAG
AUUAGCdAdAdAdAdAdAdAdAdAdAdA[Biotin-dT]dAdAdAd
AdAdAdAdAdA[Sp9][Sp9][Sp9]dAdCdC[Puromycin]
xRNA v0.1.1-Puromycin:
SEQ ID NO 23
cap1-GGGAGAGCCACCAUGGUGAGCGGCUGGCGGCUGUUCAAGA
AGAUUAGCdAdAdAdAdAdAdAdAdAdAdA[Biotin-dT]
dAdAdAdAdAdAdAdAdA[Sp9][Sp9][Sp9]dAdCdC

24 hours prior to transfection, A549 cells were seeded into an opaque 96-well clear-bottom plate (10,000 cells per well). At the time of transfection, cells were transfected with 50 ng of RNA or RNA-DNA chimera (xRNA) per well, using 0.25 ul Lipofectamine MessengerMax following standard conditions. At 6 hours post-transfection, luminescence activity was detected using the Nano-Glo HiBiT Lytic Detection System (Promega) using standard conditions. Briefly, 1 volume of complete lysis buffer (containing LgBiT and substrate) was added to each well, and the plate was mixed on an orbital shaker for 10 minutes. The Luminescence signal was then detected using a CLARIOstar plate reader.

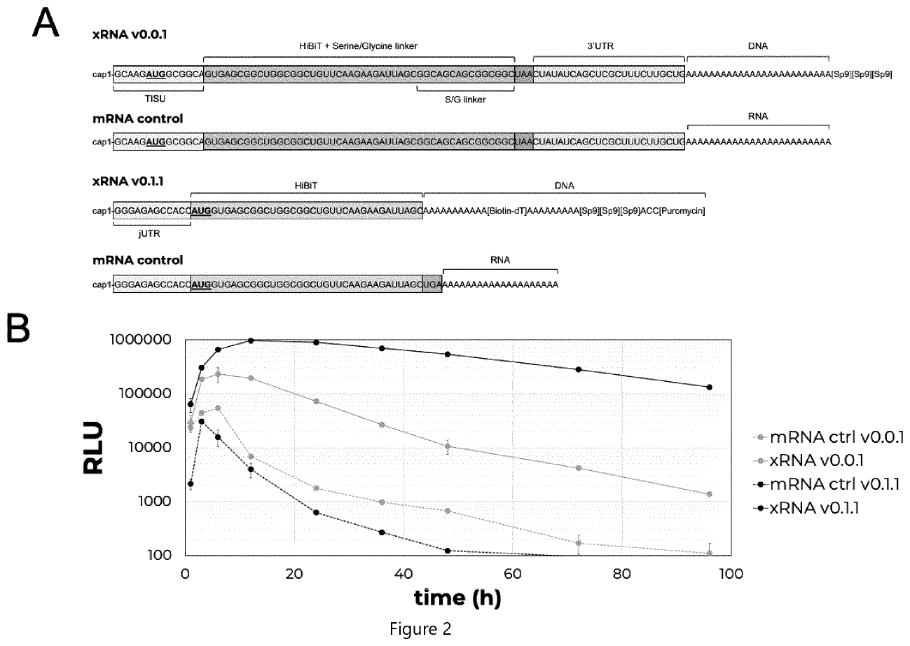

Based on the initial results as depicted in FIG. 1 B showing enhanced luminescence signal from cells transfected with the HiBiT chimeras relative to control RNAs, we performed an extended time course in which the luminescence signal was detected up to 96 hours post-transfection (FIG. 2 B). In this experiment, we monitored luminescence from cells transfected with xRNA v0.1.1+Puromycin, or a control (‘naked’) RNA. As before, A549 cells were seeded in opaque 96-well clear-bottom plates 24 hours prior to transfection. Cells were transfected with 25 ng of each construct using 0.125 ul Lipofectamine MessengerMax following standard conditions. Luminescence was monitored at 1 h, 3 h, 6, 12 h, 24 h, 36 h, 48 h, 72 h and 96 h post-transfection.

In conclusion, mRNAs encoding HiBiT with fully synthetic 3′ termini are efficiently translated in transfected cells showing that a canonical poly(A) sequence of an mRNA can be replaced by synthetic 3′ termini according to the invention. Additionally, these data show that not only that a poly(A) sequence can be replaced but also that stop codon of an ORF and/or a downstream 3′UTR can be dispersible when replaced by the synthetic 3′ termini according to the invention.

The HiBiT peptide levels produced from cells transfected with a 3′end modified RNA (or ‘xRNA’) are higher and with significantly enhanced duration over a 96 hour time course relative to a non-modified control (HiBiT RNA+poly(A)).

### Example 2

### Long-Term Stabilization of RNA Using Non-Poly(A) DNA Sequences

Based on our findings disclosed in Example 1, we next tested whether a non-poly(A) DNA sequence could achieve similarly strong effects on HiBiT long-term activity.

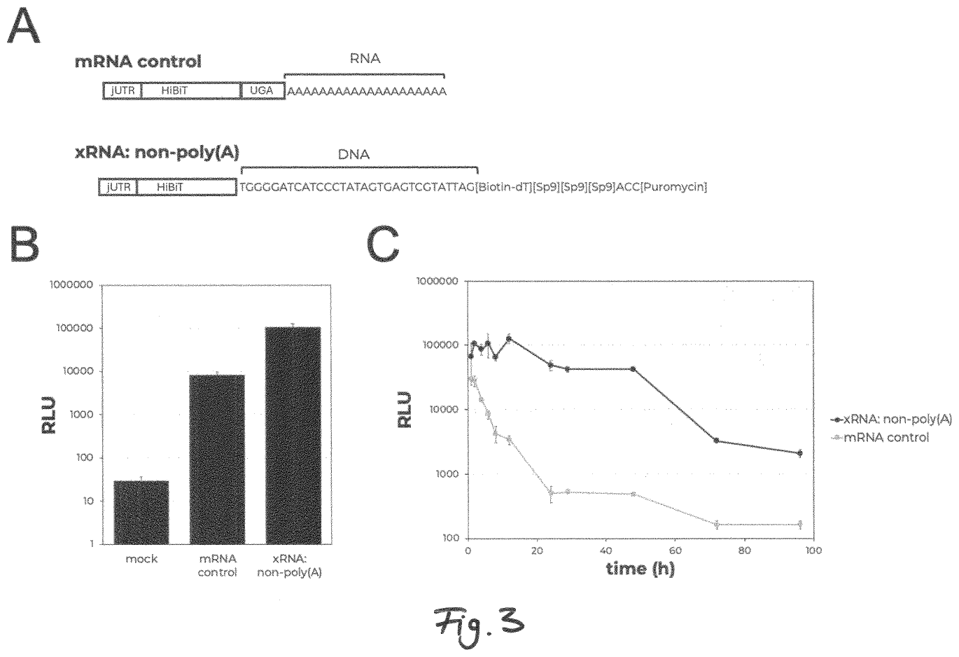

We generated a new xRNA construct termed ‘xRNA: non-Poly(A)’ by ligating an in vitro transcribed RNA encoding HiBiT flanked by 7aa Glycine/Serine linkers, to a DNA oligo containing a 31 nt non-Poly(A) sequence, plus additional modifications including biotin-dT, Spacer9 and Puromycin (FIG. 3A, schematic diagram of construct). The ‘xRNA: non-Poly(A)’ construct does not have a stop codon. A control mRNA encoding HiBiT flanked by 7aa Glycine/Serine linkers, with a stop codon and with a 20 nt Poly(A) RNA tail, was also generated by in vitro transcription, termed ‘mRNA control’. RNA was in vitro transcribed using HiScribe T7 High Yield RNA synthesis kit (NEB) according to manufacturer's instructions. All constructs were capped with Vaccinia capping enzyme and mRNA Cap 2′-O-Methyltransferase (NEB) to produce a Cap-1 structure.

Reverse Template for IVT (DNA Sequence; for Generating xRNA: NonPoly(A)):

SEQ ID NO 24:
GGTGGATCTACTCTGCTATTTTTGCGGGCTTGTAACCGCTTTATTGCCGA
AGCAATAAAGCTGCATTAGATCCACCACCACTGGAACCGCTAATCTTCTT
GAACAGCCGCCAGCCGCTCACACCAGAACTTCCTCCACCTGACATGGTGG
CTCTCCCTATAGTGAGTCGTATTA

DNA-Puromycin Oligo (for Generating xRNA: NonPoly(A))(L007):

SEQ ID NO 25 (for oligo until Sp9 linker):
dTdGdGdGdGdAdTdCdAdTdCdCdCdTdAdTdAdGdTdGd
AdGdTdCdGdTdAdTdTdAdG[Biotin-dT][Sp9][Sp9]
[Sp9]dAdCdC[Puromycin]

Reverse Template for IVT (DNA Sequence; IVT Product Corresponds to Sequence of mRNA Control):

SEQ ID NO 26
TTTTTTTTTTTTTTTTTTTTTCAAGATCCACCACCACTGGAACCGCTA
ATCTTCTTGAACAGCCGCCAGCCGCTCACACCAGAACTTCCTCCACCT
GACATGGTGGCTCTCCCTATAGTGAGTCGTATTA
mRNA control:
SEQ ID NO 27
cap1-GGGAGAGCCACCAUGUCAGGUGGAGGAAGUUCUGGUGUGAG
CGGCUGGCGGCUGUUCAAGAAGAUUAGCGGUUCCAGUGGUGGUGGA
UCUUGAAAAAAAAAAAAAAAAAAAAA

Similarly, to previous experiments, 24 hours prior to transfection, A549 cells were seeded into an opaque 96-well clear-bottom plate (10,000 cells per well). At the time of transfection, cells were transfected with 50 ng of RNA or RNA-DNA chimera per well, using 0.25 ul Lipofectamine MessengerMax following standard conditions. Luminescence activity was measured over a 96-hour time course (FIGS. 3B and 3C). Luminescence was detected using the Nano-Glo HiBiT Lytic Detection System (Promega) using standard conditions. Briefly, 1 volume of complete lysis buffer (containing LgBiT and substrate) was added to each well, and the plate was mixed on an orbital shaker for 10 minutes. The Luminescence signal was then detected using a CLARIOstar plate reader.

Results are shown in FIGS. 3 B and C. In conclusion, RNA with a non-poly(A)-DNA sequence at the 3′end is more efficiently translated than a corresponding RNA control with a conventional poly(A)-RNA tail, similar to effects observed for constructs with a poly(A)-DNA sequence (see also Example 1).

HiBiT activity from RNA with a modified 3′end that is not a poly(A)-RNA tail can be measured >10-fold above background for up to at least 96 h post-transfection, while an equivalent mRNA control reaches background levels by 72 h.

### Example 3

### Non-Natural RNA Elements on the 3′End of mRNA Enhance Expression In Vitro

To measure the effects of synthetic 3′ terminus modifications on mRNA functionality, a small-scale screen was conducted using HiBiT as a model mRNA. Several 3′ends comprising a fixed 3′UTR sequence and followed by natural or non-natural RNA elements of various length and nature, were enzymatically ligated onto in vitro generated, capped (cap1) HiBiT mRNA. The functionality of ligation products was assessed in vitro in A549 and Hek293T cells.

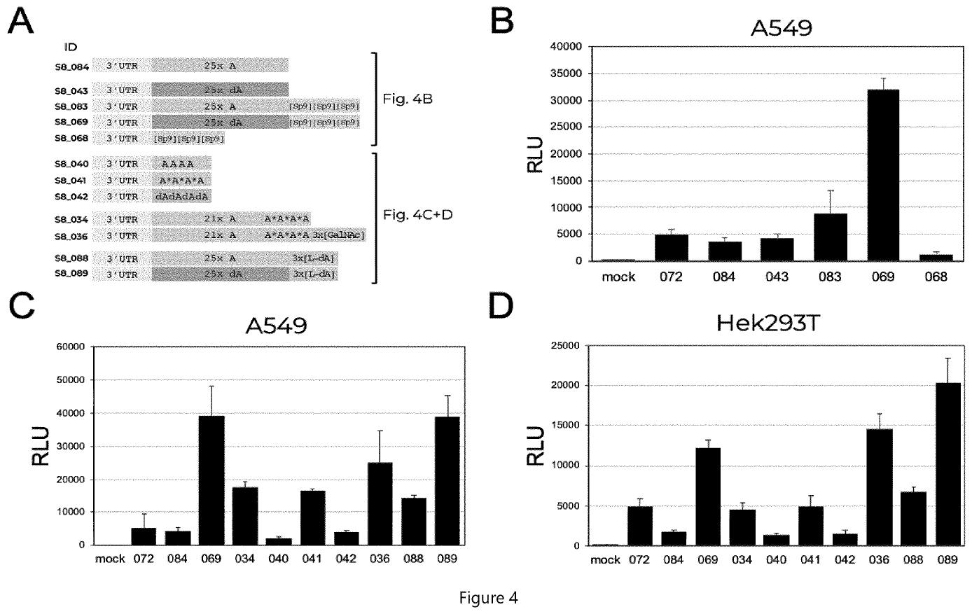

An illustration of all constructs tested can be found in FIG. 4A. Below are the oligos used to generate these constructs via RNA-RNA ligation.

### Oligos Used:

Product of in vitro transcription: HiBiT mRNA consisting of 5′UTR, HiBiT CDS and partial 3′UTR. This sequence was further enzymatically capped with cap1 using the NEB Vaccinia capping system including 2′O-Methyltransferase:

S8_070 (Product after In Vitro Transcription): (SEQ ID NO 28:

cap1-GCAAGAUGGCGGCAGUGAGCGGCUGGCGGCUGUUCAAGAAG
AUUAGCGGCAGCAGCGGCGGCUAACUAUAUCA-

### Splint for RNA-RNA Ligations: S8_066 (SEQ ID NO 29):

AGCGAGCTGATATAGTTAGCCGCCGCTGCT

S8_072 (SEQ ID NO 30 (Product after In Vitro Transcription; Sequence Corresponds to S8_070 with 58_033 Ligated on):

Cap1-GCAAGAUGGCGGCAGUGAGCGGCUGGCGGCUGUUCAAGAAG
AUUAGCGGCAGCAGCGGCGGCUAACUAUAUCA-AAAAAAAAAAAAA
AAAAAAAAAAAA

Tables provided below (Table 4 to 6) includes numerous polynucleotide modification structures that comprise a natural or unnatural polynucleotide. These structures are shown in 5′ to 3′ orientation and were prepared by phosphoramidite synthesis, and are commercially available from custom oligonucleotide vendors such as Integrated DNA Technologies (Coral ville, Iowa, USA), or GeneLink (Orlando, Florida, USA). There are numerous of non-standard phosphoramidite monomer unit “building blocks” published and commercially available from custom polynucleotide vendors that can be easily incorporated into custom synthesized polynucleotides. Many of these non-standard monomer units are classified as spacers (e.g., “iSp”), and affinity tags (e.g., “[Bio-dT]”). All polynucleotide modification structures in the tables below are described using well-known polynucleotide synthesis nomenclature to indicate the non-standard monomer units. (See e.g., the web-site of Integrated DNA Technologies (IDT) at https://eu.idtdna.com/site/catalog/Modifications/GetAllMods, or GenLink at http://www.genelink.com/newsite/products/OligoModifications.asp for further details of commonly used oligonucleotide nomenclature.) For example, non-standard monomer units are enclosed in forward slashes (“/”) or in brackets (“[” and “]”), and an asterisk “*” between units indicates a phosphorothioate linkage. A key of further selected abbreviations is included in tables below.

TABLE 4
3′end oligos ligated onto S8_070

| ID | SEQ ID NO | Polynucleotide sequence | |
|---|---|---|---|
| (3′terminus) | of oligo | (5′-> 3′) | Description |
| S8_084 | SEQ ID NO | /5Phos/GCUCGCUUUCUUGCUGAAAAA | SEQ ID NO 31 |
| | 31 | AAAAAAAAAAAAAAAAAAAA | (3′UTR followed by |
| | | | 25 A (RNA)) |
| S8_043 | SEQ ID NO | /5Phos/GCUCGCUUUCUUGCUGdAdAd | SEQ ID NO 32 |
| | 32 | AdAdAdAdAdAdAdAdAdAdAdAdAdAd | (3′UTR followed by |
| | | AdAdAdAdAdAdAdAdA | 25 dA (DNA)) |
| S8_083 | SEQ ID NO | /5Phos/GCUCGCUUUCUUGCUGAAAAA | SEQ ID NO X33 |
| | 33 | AAAAAAAAAAAAAAAAAAAA[Sp9] | linked to 3x Spacer9 |
| | | [Sp9][Sp9] | (3′UTR followed by |
| | | | 25 A (RNA) and |
| | | | 3xSpacer9) |
| S8_069 | SEQ ID NO | /5Phos/GCUCGCUUUCUUGCUGdAdAd | SEQ ID NO 34 linked |
| | 34 | AdAdAdAdAdAdAdAdAdAdAdAdAdAd | to 3x Spacer9 |
| | | AdAdAdAdAdAdAdAdA[Sp9] | (3′UTR followed by |
| | | [Sp9][Sp9] | 25 dA (DNA) and |
| | | | 3xSpacer9) |
| S8_068 | SEQ ID NO | /5Phos/GCUCGCUUUUUGCUG[Sp9][S | SEQ ID NO 35 linked |
| | 35 | p9][Sp9] | to 3x Spacer9 |
| | | | (3′UTR followed by |
| | | | 3xSpacer9) |
| S8_040 | SEQ ID NO | /5Phos/GCUCGCUUUCUUGCUGAAAA | SEQ ID NO 36 |
| | 36 | | (3′UTR followed by |
| | | | 4 A (RNA)) |
| S8_041 | SEQ ID NO | /5Phos/GCUCGCUUUCUUGCUGA*A*A* | SEQ ID NO 37, |
| | 37 | A | wherein nucleobase |
| | | | at position 18, 19, |
| | | | and 20 are linked to |
| | | | the previous |
| | | | nucleobase by |
| | | | phosphorothioate |
| | | | bonds |
| | | | (3′UTR followed by |
| | | | 4 A (RNA) with |
| | | | interspersed |
| | | | phosphorothioate |
| | | | bonds) |
| S8_042 | SEQ ID NO | /5Phos/GCUCGCUUUCUUGCUGdAdAd | SEQ ID NO 38 |
| | 38 | AdA | (3′UTR followed by |
| | | | 4 dA (DNA)) |
| S8_034 | SEQ ID NO | /5Phos/GCUCGCUUUCUUGCUGAAAAA | SEQ ID NO 39, |
| | 39 | AAAAAAAAAAAAAAAAA*A*A*A | wherein nucleobase |
| | | | at position 39, 40, |
| | | | and 41 are linked to |
| | | | the previous |
| | | | nucleobase by |
| | | | phosphorothioate |
| | | | bonds |
| | | | (3′UTR followed by |
| | | | 21 A and 4 A (RNA) |
| | | | with Interspersed |
| | | | phosphorothioate |
| | | | bonds) |
| S8_036 | SEQ ID NO | /5Phos/GCUCGCUUUCUUGCUGAAAAA | SEQ ID NO 39, |
| | 39 | AAAAAAAAAAAAAAAAA*A*A*A | wherein nucleobase |
| | | [GaINAc][GalINAc][GalNAc] | at position 39, 40, |
| | | | and 41 are linked to |
| | | | the previous |
| | | | nucleobase by |
| | | | phosphorothioate |
| | | | bonds, and the |
| | | | terminal nucleobase |
| | | | is linked to 3x linear |
| | | | GalNAcs |
| | | | (3′UTR followed by |
| | | | 21 A and 4 A (RNA) |
| | | | with interspersed |
| | | | phosphorothioate |
| | | | bonds and 3x linear |
| | | | GalNAcs) |
| S8_088 | SEQ ID NO | /5Phos/GCUCGCUUUCUUGCUGAAAAA | SEQ ID NO 40, |
| | 40 | AAAAAAAAAAAAAAAAAAAA[L-dA] | wherein nucleobase |
| | | [L-dA][L-dA] | 41, 42, and 43 are |
| | | | [L-dA] |
| | | | (3′UTR followed by |
| | | | 25 A (RNA) and 3xL- |
| | | | DNA A) |
| S8_089 | SEQ ID NO | /5Phos/GCUCGCUUUCUUGCUGdAdAd | SEQ ID NO 41, |
| | 41 | AdAdAdAdAdAdAdAdAdAdAdAdAdAd | wherein nucleobase |
| | | AdAdAdAdAdAdAdAdA[L-dA][L-dA] | 41, 42, and 43 are |
| | | [L-dA] | [L-dA] |
| | | | (3′UTR followed by |
| | | | 25 dA (DNA) and |
| | | | 3xL-DNA A) |

SEQ ID NOs shown correspond to nucleic acid section of molecules depicted in “Polynucleotide sequence (5′->3′)” column of Table 4.

### Key for Non-Standard Sequence Elements:

/5Phos/=5′ monophosphate
/iSp9/=internal Spacer9
/3Sp9/=3′ terminal Spacer9
/iBiodT/=internal Biotin-dT
*=phosphorothioate bond
dA=2′-deoxy adenosine (DNA)
[GalNAc]=N-acetylgalactosamine
[L-dA]=beta-L-2′-deoxy adenosine

Various 3′ends (see Table 4) were ligated onto in vitro transcribed and capped (cap1) HiBiT mRNA (cap1-S8_070) using T4 RNA ligase 2 (NEB) via RNA-RNA splint ligation (final products shown in Table 5). Resulting ligation products were PAGE purified from 15% TBE-UREA gels (Novex) and purified using Zymo RNA clean & concentrator 25 columns. 50 ng of each RNA product were transfected in triplicates in 96-well plates into A549 (10.000 cells/well) or Hek293T cells (15.000 cells/well) using 0.25 ul Lipofectamine MessengerMax using standard conditions. HiBiT activity was assessed 6 h post-transfection using the NanoLuc HiBiT lytic detection system (Promega) according to manufacturer's instructions. Luminescence was measured on a BMG Labtech Clariostar plus microplate reader.

TABLE 5
List of ligation products of 3′ends listed in Table 4 ligated
onto cap1-S8_070

| | SEQ ID NOs | Final ligation product (5′-> 3′) |
|---|---|---|
| | of fused | (fused oligos, ligation |
| ID (mRNA) | oligos | site marked by -) |
| S8_084 | SEQ ID NO | GCAAGAUGGCGGCAGUGAGCGGCUGGCGGCUGUUCAAGAA |
| | 28- | GAUUAGCGGCAGCAGCGGCGGCUAACUAUAUCA-GCUCGCU |
| | SEQ ID NO | UUCUUGCUGAAAAAAAAAAAAAAAAAAAAAAAAA |
| | 31 | |
| S8_043 | SEQ ID NO | GCAAGAUGGCGGCAGUGAGCGGCUGGCGGCUGUUCAAGAA |
| | 28-32 | GAUUAGCGGCAGCAGCGGCGGCUAACUAUAUCA-GCUCGCU |
| | | UUCUUGCUGdAdAdAdAdAdAdAdAdAdAdAdAdAdAdAdAd |
| | | AdAdAdAdAdAdAdAdA |
| S8_083 | SEQ ID NO | GCAAGAUGGCGGCAGUGAGCGGCUGGCGGCUGUUCAAGAA |
| | 28- | GAUUAGCGGCAGCAGCGGCGGCUAACUAUAUCA-GCUCGCU |
| | SEQ ID NO | UUCUUGCUGAAAAAAAAAAAAAAAAAAAAAAAAA[Sp9][Sp9] |
| | 33 | [Sp9] |
| S8_069 | SEQ ID NO | GCAAGAUGGCGGCAGUGAGCGGCUGGCGGCUGUUCAAGAA |
| | 28- | GAUUAGCGGCAGCAGCGGCGGCUAACUAUAUCA-GCUCGCU |
| | SEQ ID NO | UUCUUGCUGdAdAdAdAdAdAdAdAdAdAdAdAdAdAdAdAd |
| | 34 | AdAdAdAdAdAdAdAdA[Sp9][Sp9][Sp9] |
| S8_068 | SEQ ID | GCAAGAUGGCGGCAGUGAGCGGCUGGCGGCUGUUCAAGAA |
| | NO | GAUUAGCGGCAGCAGCGGCGGCUAACUAUAUCA-GCUCGCU |
| | 28- | |
| | SEQ ID NO | |
| | 35 | |
| S8_040 | SEQ ID NO | GCAAGAUGGCGGCAGUGAGCGGCUGGCGGCUGUUCAAGAA |
| | 28- | GAUUAGCGGCAGCAGCGGCGGCUAACUAUAUCA-GCUCGCU |
| | SEQ ID NO | UUCUUGCUGAAAA |
| | 36 | |
| S8_041 | SEQ ID NO | GCAAGAUGGCGGCAGUGAGCGGCUGGCGGCUGUUCAAGAA |
| | 28- | GAUUAGCGGCAGCAGCGGCGGCUAACUAUAUCA-GCUCGCU |
| | SEQ ID NO | UUCUUGCUGA*A*A*A |
| | 37 | |
| S8_042 | SEQ ID NO | GCAAGAUGGCGGCAGUGAGCGGCUGGCGGCUGUUCAAGAA |
| | 28- | GAUUAGCGGCAGCAGCGGCGGCUAACUAUAUCA-GCUCGCU |
| | SEQ ID NO | UUCUUGCUGdAdAdAdA |
| | 38 | |
| S8_034 | SEQ ID NO | GCAAGAUGGCGGCAGUGAGCGGCUGGCGGCUGUUCAAGAA |
| | 28- | GAUUAGCGGCAGCAGCGGCGGCUAACUAUAUCA- |
| | SEQ ID NO | GCUCGCUUUCUUGCUGAAAAAAAAAAAAAAAAAAAAAA*A* |
| | 39 | A*A |
| S8_033 | SEQ ID NO | GCAAGAUGGCGGCAGUGAGCGGCUGGCGGCUGUUCAAGAA |
| | 30 | GAUUAGCGGCAGCAGCGGCGGCUAACUAUAUCAGCUCGCU |
| | | UUCUUGCUGAAAAAAAAAAAAAAAAAAAAAAAAA |
| S8_036 | SEQ ID NO | GCAAGAUGGCGGCAGUGAGCGGCUGGCGGCUGUUCAAGAA |
| | 28- | GAUUAGCGGCAGCAGCGGCGGCUAACUAUAUCA- |
| | SEQ ID NO | GCUCGCUUUCUUGCUGAAAAAAAAAAAAAAAAAAAAAA*A* |
| | 39 | A*A[GalNAc][GalNAc][GalNAc] |
| S8_088 | SEQ ID NO | GCAAGAUGGCGGCAGUGAGCGGCUGGCGGCUGUUCAAGAA |
| | 28- | GAUUAGCGGCAGCAGCGGCGGCUAACUAUAUCA- |
| | SEQ ID NO | GCUCGCUUUCUUGCUGAAAAAAAAAAAAAAAAAAAAAAAAA |
| | 40 | [L-dA][L-dA][L-dA] |
| S8_089 | SEQ ID NO | GCAAGAUGGCGGCAGUGAGCGGCUGGCGGCUGUUCAAGAA |
| | 28- | GAUUAGCGGCAGCAGCGGCGGCUAACUAUAUCA- |
| | SEQ ID NO | GCUCGCUUUCUUGCUGdAdAdAdAdAdAdAdAdAdAdAdAdA |
| | 41 | dAdAdAdAdAdAdAdAdAdAdAdA[L-dA][L-dA][L-dA] |

SEQ ID NOs shown correspond to nucleic acid section of molecules depicted in “Final ligation product” column of Table 5.

Synthetic 3′ terminus modifications lead to enhanced HiBiT activity compared to unmodified controls, irrespective of whether controls were generated by in vitro transcription or via RNA-RNA ligations.

Furthermore, there is no requirement for a poly(A) RNA tail for efficient HiBiT translation: The poly(A) RNA tail can be fully replaced with a poly(A) DNA sequence (or other non-poly(A) DNA sequences—see Example 2), leading to a significant increase in HiBiT activity.

Additionally, we show that effect size may be linked to 3′end length. Very short 3′ends do exhibit the same level of stabilization, however this was not fully explored. S8_041 (FIG. 4) suggests that with further stabilization using e.g. phosphorothioate bonds, 4xA could be sufficient to achieve high translational yield.

We also show a combinatorial effect of non-natural sequence elements: combining several non-natural elements leads to signal enhancement higher than expected based on the effects of individual elements (compare 043, 068 with 069).

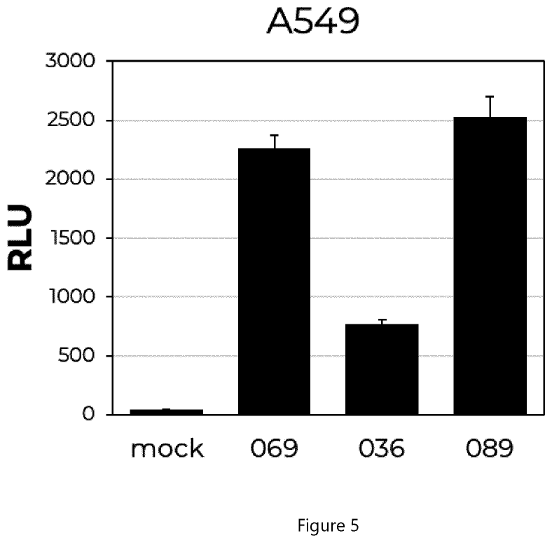

The results obtained in this 3′end screen are robust in 2 cell lines of diverse origin: A549 (lung epithelial cells), which are sensitive to innate immune activation, and Hek293T (Human embryonic kidney cells), which are impaired in certain innate immune pathways. The high correlation of results (R2=0.9) shows that constructs tested here do not display any inherent immunogenicity or toxicity. Similar patterns remain after 48 h (FIG. 5) as observed after 6 h, suggesting longer term stability can be achieved with different 3′end modifications. Notably, the lesser performing construct (S8_036) contains an RNA poly(A) sequence, while both better performing constructs contain DNA poly(A) sequences. This confirms results in FIG. 4 suggesting that signal enhancement is conferred by a combination of both poly-DNA sequences and additional non-natural elements. When comparing the best performing construct containing 25dA+3×Sp9 at its 3′end (S8_069, or xRNA v0.01 in FIG. 2) against xRNA v0.1.1 (see example 1), which is comprised of a shorter CDS (13 vs 20 amino acids) and does not contain a 3′UTR or stop codon, the synergistic effect of various non-natural modifications becomes even more apparent: At 96 h post-transfection, while HiBiT activity of xRNA v0.1.1 remains about 1000-fold above background and its corresponding mRNA control, HiBiT activity of xRNA v0.0.1 has dropped to about 10-fold above background.

### Example 4

### A Synthetic 3′ Terminus Enhances Protein and Secretion Levels of a Therapeutic Peptide: GLP-1

In order to test whether our previous findings could be translated to a different and therapeutically relevant modality, we tested similar synthetic 3′ terminus sequences in the context of Glucagon-like peptide 1 (GLP-1), a secreted peptide.

In brief, all GLP-1 RNA oligos were generated via in vitro transcription followed by enzymatic capping with cap0 or cap1 (as indicated in figure legends) using the NEB Vaccinia capping system and mRNA Cap 2′-O-Methyltransferase (for cap1 only). Oligos with chimeric 3′ends were generated by RNA-RNA ligations of an in vitro generated 5′end (in vitro transcription followed by capping),—consisting of 5′ UTR, signal peptide, furin cleavage site, GLP-1 CDS and short 3′UTR—to a chemically synthesized 3′end oligo (synthetic 3′ terminus).

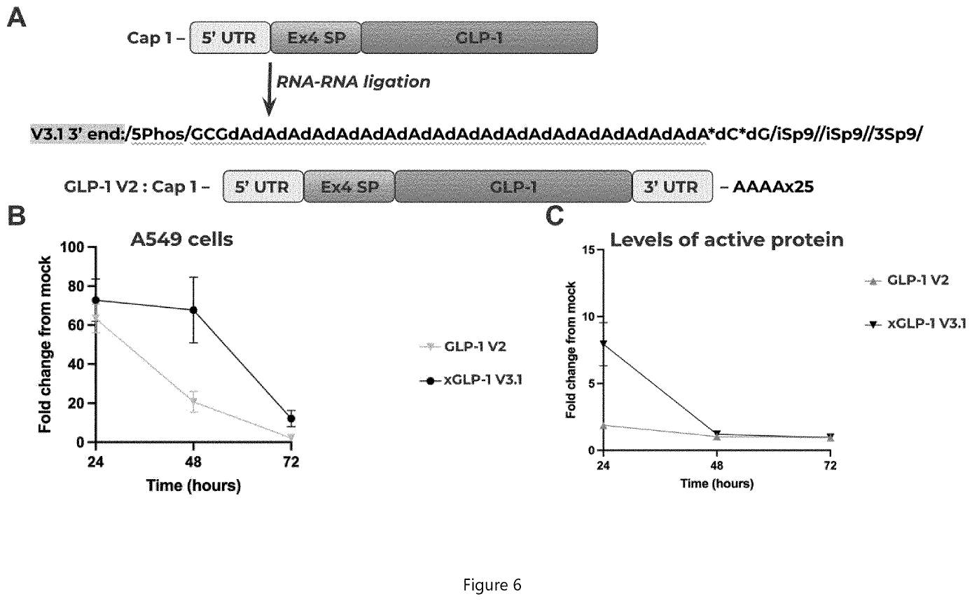

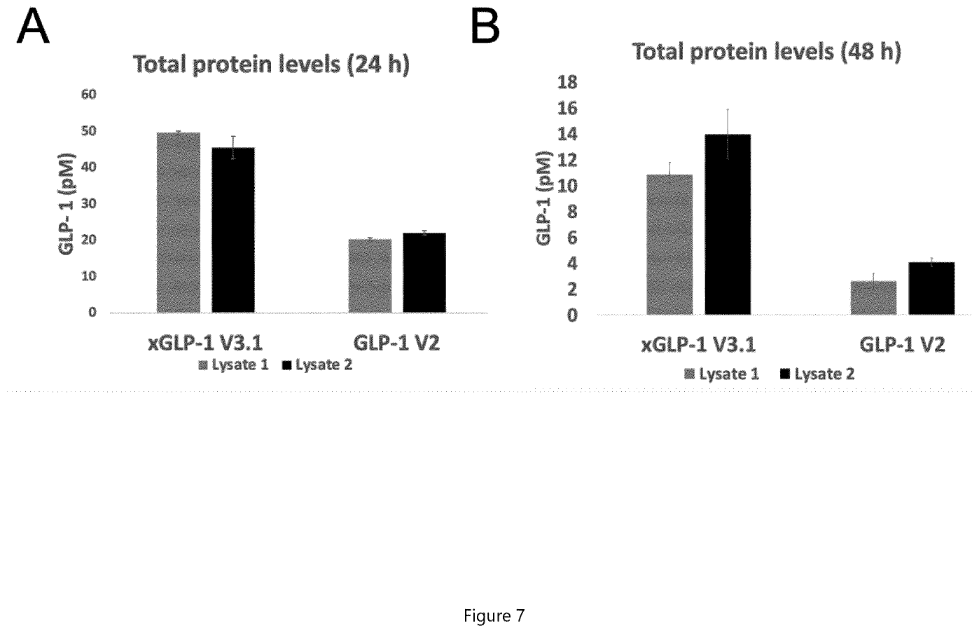

10 ng (FIGS. 6, 7 and 8) of each RNA product were transfected (reverse transfection) in triplicates in 96-well plates into A549 cells in suspension (30,000 cells/well) or 200 ng of each RNA product were transfected (reverse transfection) into HepG2 cells in suspension (30,000 cells/well). GLP-1 activity was assessed 24 h post-transfection using the Bright-Glo Luciferase Assay System (Promega) according to manufacturer's instructions and reading luminescence on a BMG Labtech Clariostar plus microplate reader.

Human GLP-1 (7-36) SimpleStep ELISA Kit (ab184857, Abcam) was used to assess active GLP-1 protein levels according to manufacturer's instructions and Mercodia Total GLP-1 ELISA Kit (Cat number: 10-1278-01) was used to assess total GLP-1 protein levels (detection of GLP-1 9-36 and 7-36) according to manufacturer's instructions.

TABLE 6
Sequences of DNA templates for in vitro transcription, modified
3′end RNA oligos and DNA splints for ligation.

| Name of the | | | |
|---|---|---|---|
| ligation | GLP-1 Twister sequences | | |
| product | (DNA template for IVT) | Modified 3′ ends (DNA/RNA) | DNA splint |
| xGLP-1 V3 | SEQ ID NO 42: | SEQ ID NO 46: | SEQ ID NO |
| | TAATACGACTCACTATAGCA | /5Phos/GCGdAdAdAdAdAdAdAdA | 50: |
| | AGATGGCGGCAATGAAGTG | dAdAdAdAdAdAdAdAdAdAdAdAd | TTTTTTTTTT |
| | GGTGACCTTCATCAGCCTGC | AdAdAdAdAdA/iSp9//iSp9//3Sp9/ | TTCGCTTAG |
| | TGTTCCTGTTCAGCAGCGCC | | CCTCTGCCT |
| | TACAGCCATGGAGAGGGCA | | TT |
| | CCTTTACCTCTGATGTGTCC | | |
| | AGCTACCTGGAAGGCCAGG | | |
| | CTGCCAAAGAGTTTATCGCC | | |
| | TGGCTGGTCAAAGGCAGAG | | |
| | GCTAAaatgcagctttattgcttcg | | |
| | gcaataaagcggttacaagcccgca | | |
| | aaaatagcagagtaTAGCCTCT | | |
| xGLP-1 V3.1 | SEQ ID NO 43: | SEQ ID NO 47: | SEQ ID NO |
| | ATAGTGGATTGGCCAGGTCC | /5phos/GCGdAdAdAdAdAdAdAdA | 51: |
| | ATTTTAATACGACTCACTAT | dAdAdAdAdAdAdAdAdAdAdAdA* | TTTTTTTTTT |
| | AGCAAGATGGCGGCAATGA | C*G/iSp9//iSp9//3Sp9/ | TTTTTTTCG |
| | AGATCATCCTGTGGCTGTGT | | CGCTAATCT |
| | GTGTTCGGCCTGTTCCTGGC | | TTTAGCCTC |
| | CACCCTGTTCCCCATCAGCT | | TGC |
| | GGCAGAAGCGCATCAAGCG | | |
| | CCATGGAGAGGGCACCTTT | | |
| | ACCTCTGATGTGTCCAGCTA | | |
| | CCTGGAAGGCCAGGCTGCC | | |
| | AAAGAGTTTATCGCCTGGCT | | |
| | GGTCAAAGGCAGAGGCTAA | | |
| | AAGATTAGCAATGCAGCTTT | | |
| | ATTGCTTCGGCAATAAAGCG | | |
| | GTTACAAGCCCGCAAAAAT | | |
| | AGCAGAGTACTAATCTT | | |
| xGLP-1 | SEQ ID NO 44: | SEQ ID NO 48: | SEQ ID NO |
| V3.2.1(P03) | ATAGTGGATTGGCCAGGTCC | /5phos/GCG/iBiodT/dAdAdAdAdA | 52: |
| | ATTTTAATACGACTCACTAT | dAdAdAdAdAdAdAdAdAdAdAdAd | TTTTTTTTTT |
| | AGCAAGATGGCGGCAATGA | | |
| | AGATCATCCTGTGGCTGTGT | | |
| | GTGTTCGGCCTGTTCCTGGC | | |
| | CACCCTGTTCCCCATCAGCT | | |
| | GGCAGAAGCGCATCAAGCG | | |
| | CCATGGAGAGGGCACCTTT | | |
| | ACCTCTGATGTGTCCAGCTA | | |
| | CCTGGAAGGCCAGGCTGCC | AdAdA*C*G/iSp9//iSp9//3Sp9/ | TTTTTTACG |
| | AAAGAGTTTATCGCCTGGCT | | CGCTAATCT |
| | GGTCAAAGGCAGAGGCTAA | | TTTAGCCTC |
| | AAGATTAGCAATGCAGCTTT | | TGC |
| | ATTGCTTCGGCAATAAAGCG | | |
| | GTTACAAGCCCGCAAAAAT | | |
| | AGCAGAGTACTAATCTT | | |
| xGLP-1 | SEQ ID NO 45: | SEQ ID NO 49: | SEQ ID NO 53: |
| V3.2.2(P15) | ATAGTGGATTGGCCAGGTCC | /5phos/GCGdAdAdAdAdAdAdAdA | |
| | ATTTTAATACGACTCACTAT | dAdAdA/iBiodT/dAdAdAdAdAdAd | TTTTTATTTT |
| | AGCAAGATGGCGGCAATGA | AdAdA*C*G/iSp9//iSp9//3Sp9/ | TTTTTTTCG |
| | AGATCATCCTGTGGCTGTGT | | CGCTAATCT |
| | GTGTTCGGCCTGTTCCTGGC | | TTTAGCCTC |
| | CACCCTGTTCCCCATCAGCT | | TGC |
| | GGCAGAAGCGCATCAAGCG | | |
| | CCATGGAGAGGGCACCTTT | | |
| | ACCTCTGATGTGTCCAGCTA | | |
| | CCTGGAAGGCCAGGCTGCC | | |
| | AAAGAGTTTATCGCCTGGCT | | |
| | GGTCAAAGGCAGAGGCTAA | | |
| | AAGATTAGCAATGCAGCTTT | | |
| | ATTGCTTCGGCAATAAAGCG | | |
| | GTTACAAGCCCGCAAAAAT | | |
| | AGCAGAGTACTAATCTT | | |

Key:
/5Phos/ = 5′ monophosphate
/iSp9/ = internal Spacer9
/3Sp9/ = 3′ terminal Spacer9
/iBiodT/ = internal Biotin-dT
*= phosphorothioate bond
dA = 2′-deoxy adenosine (DNA)

A synthetic 3′ terminus enhances and prolongs total as well as functional GLP-1 protein levels relative to an unmodified mRNA control (FIG. 6+7):

The enhancement of functional GLP-1 production can also be observed in the context of a different GLP-1 peptide, containing a different N-terminal signal peptide compared to previous examples.

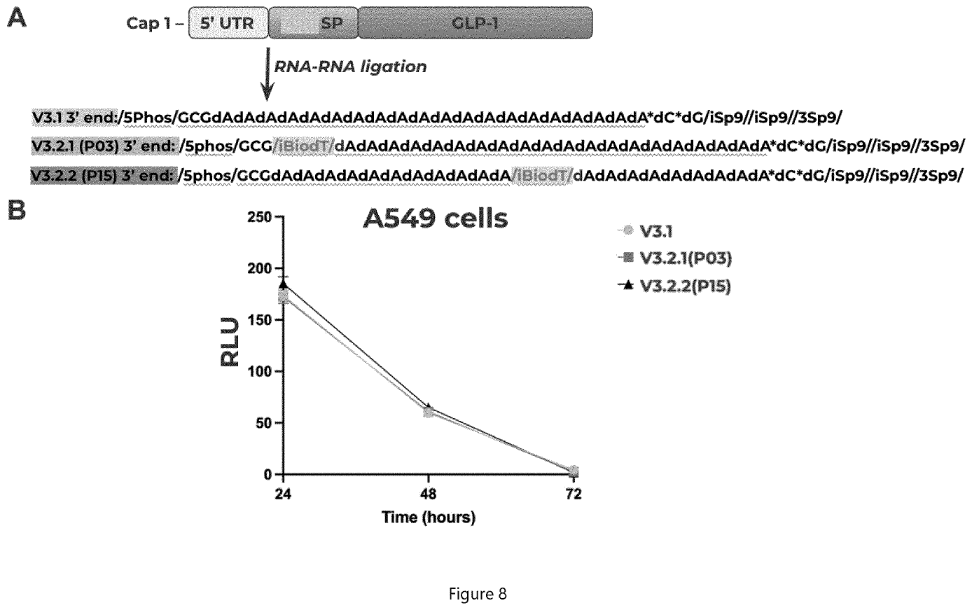

Variations of the previous synthetic 3′ terminus led to a similar enhancement in active GLP-1 production as V3.1 (FIG. 8):
Synthetic 3′ termini are functional even in context of a therapeutically relevant and 20 secreted peptide, such as GLP-1
Synthetic 3′ termini display enhanced and prolonged translatability, secretion efficiency and peptide activity as demonstrated in cell lineA549

The synthetic 3′ termini can be further modified with additional non-natural modifications, such as biotin-dT, without affecting functionality.

### Example 5

### A Non-Natural RNA Element on the 3′End of an mRNA Enhances Expression In Vivo

To test whether the in vitro results would translate into in vivo, we conducted pharmacokinetic studies in mice to address 3 specific aims:
(a) Can xRNA be detected in vivo?
(b) Do xRNA display a longer half-life compared to a corresponding mRNA control?
(c) Are there any clinical signs of toxicity/immunogenicity of xRNA?

LNP production: Lipid nanoparticles (LNPs) were prepared using the NanoAssembir Ignite+ (Precision NanoSystems) with a flow rate ratio of 3:1 and flow rate of 12 ml/min. MC3 LNPs were composed of MC3:DSPC:Chol:DMG-PEG2000 at 50:10:38.5:1.5 molar ratio. Individual lipid stocks were dissolved using absolute ethanol (99.8%). The final lipid concentration after microfluidic production was 6 mM. The aqueous solution containing mRNA was prepared using 50 mM citrate buffer pH 4.5 at the nitrogen-to-phosphate ratio (N/P) of 6.

Formulations were dialysed (MWCO 12,000-14,000 Da, GeBAflex-tube) for 10-16 hours in 1×PBS buffer at 4° C., with one complete PBS change during the dialysis.

Particle size (Z-average diameter) and polydispersity index (PDI) were measured by dynamic light scattering (DLS) in a Zetasizer Nano ZS (Malvern). The encapsulation efficiency was quantified by RiboGreen assay following manufacturer instructions. Fluorescence was measured at excitation and emission wavelengths of 483 and 530 nm.

LNP delivery: mRNA and xRNA constructs were encapsulated in MC3 LNPs and delivered to mice.

In vivo experiment: C57BL/6 J female 8-10 weeks old mice were used in this study.

Animals were weighed prior to dosing. Animals received 10 ug of cap1-mRNA and cap1-xRNA via intravenously (IV). PBS served as controls. Animals were monitored for clinical abnormalities including body weight, labored breathing, hunch posture, mobility, and discharge from eyes post 1 hr of therapeutic agent administration and daily. Liver and blood were collected at 6, 12, 24, 48, 72 & 96 hrs post dosing. HiBiT protein levels were tested in fresh liver lysate using Nano-Glo® HiBiT Lytic Detection system (Cat #N3030, Promega, WI). Serum IP-10 (Cat #IMSIP10KT, Innovative research, MI, USA) and IL-6 (Cat #88-7064-88—Thermofisher Scientific, MA, USA) levels were analyzed by ELISA. Different dilutions of serum were tested for cytokine as per manufacturer's protocol.

### Oligos Used:

### Cap1-mRNA Control (RNA):

SEQ ID NO 54 with 5′ Cap-1 Structure:

cap1-GGGAGAGCCACCAUGGUGAGCGGCUGGCGGCUGUUCAAG
AAGAUUAGCUAAAAAAAAAAAAAAAAAAAAAA

### Cap1-xRNA (RNA/DNA Chimera):

SEQ ID NO 55 with 5′ cap-1 structure, wherein nucleobase at position 69 and 70 are bound to the previous nucleobase by a phosphorothioate bond and the terminal base is linked to 3×Sp9-1×C3:

cap 1-GGGAGAGCCACCAUGGUGAGCGGCUGGCGGCUGUUCAA
GAAGAUUAGCdAdAdAdAdAdAdAdAdAdAdAdAdAdAdAdAdA
dAdAdA*dC*dG[Sp9][Sp9][Sp9][C3]

Study 1: The study was designed to determine the in vivo translation and durability of Cap1-xRNA compared to PBS (background) up to 96 hrs.

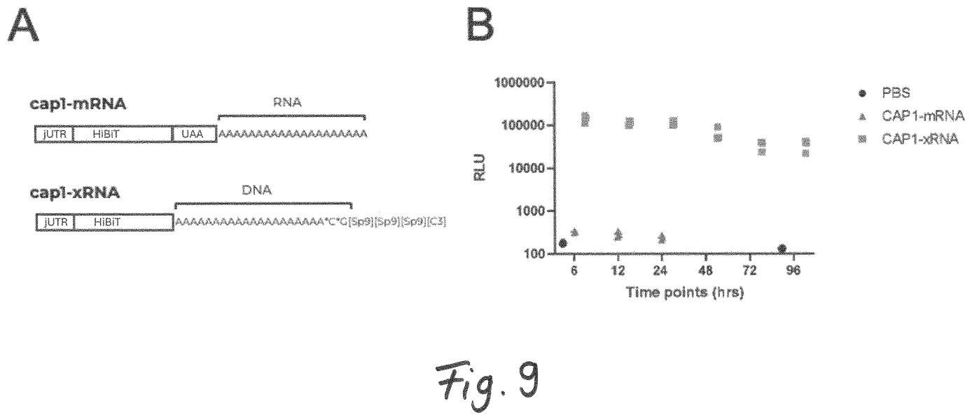

In order to assess xRNA translation in vivo, mice were injected with either LNP encapsulated cap1-xRNA or a corresponding cap1-mRNA (FIG. 9). HiBiT peptide was detected from fresh liver lysates at stated time points up to 96 h post-injection using Nano-Glo® HiBiT Lytic Detection system (Cat #N3030, Promega, WI).

Results: Comparison of cap1-xRNA against cap1-mRNA control

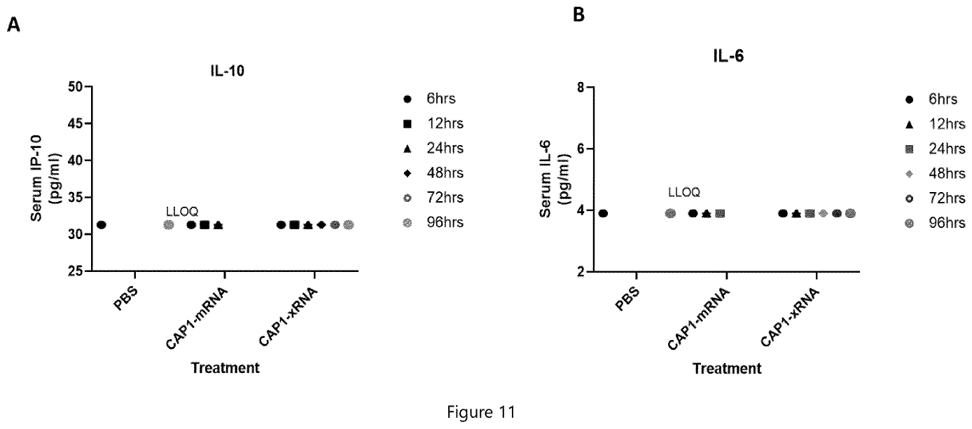

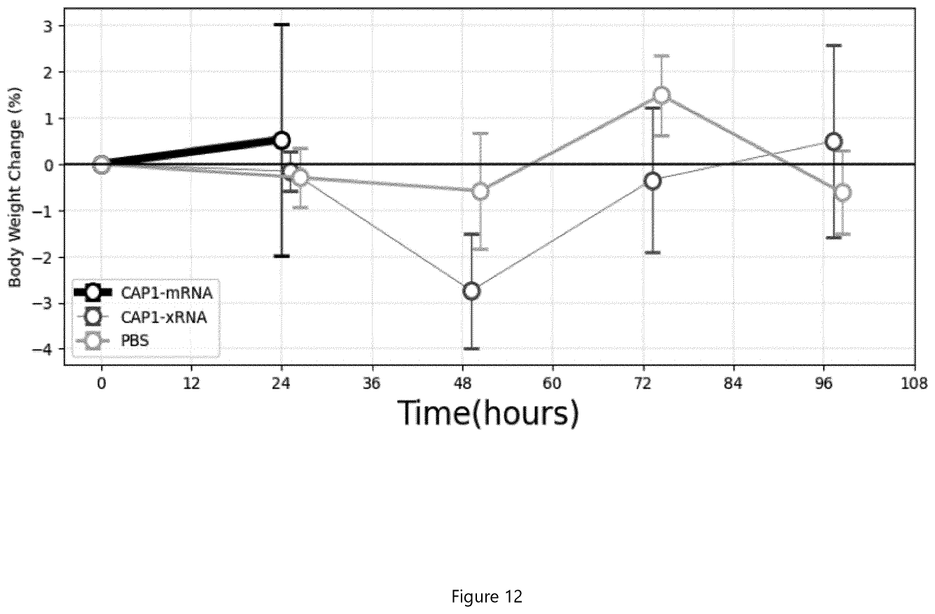

The percent body weight change is comparable to PBS controls and no remarkable effect in body weight due to cap1-xRNA administration (FIG. 12). Animals tolerated cap1-mRNA and cap1-xRNA well and no clinical abnormalities were observed. Serum IP-10 and IL-6 at different time points (6 hrs to 96 hrs) were below the lower limit of detection of commercially used ELISA kits (FIG. 11). HiBiT activity of the mRNA control was detected at background levels after 24 h while cap1-xRNA still showed >100-fold increased levels above background. Relevant samples in plot: PBS (background), cap1-mRNA and cap1-xRNA.
Study 2: The study was designed to determine the durability of Cap1-xRNA for 10 days

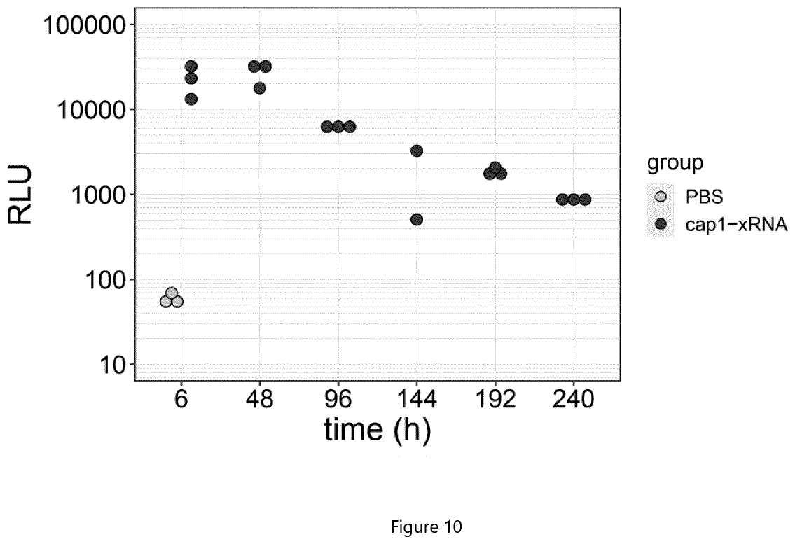

In order to better understand the translation kinetics of delivered xRNA, a long-term pharmacokinetic study to measure durability of cap1-xRNA was carried out (FIG. 10). C57BL/6J female 8-10 weeks old mice were used in this study. Animals were weighed prior to dosing. Animals received 10 μg of cap1-xRNA or xcap-xRNA via intravenous injection (IV). PBS served as controls. Animals were monitored for clinical abnormalities including body weight, labored breathing, hunch posture, mobility and discharge from eyes post 1 hr of therapeutic agent administration and daily. Liver and blood were collected at 6, 48, 96, 144, 192 & 240 hrs post dosing. HiBiT protein levels were tested in fresh liver lysate using Nano-Glo® HiBiT Lytic Detection system (Cat #N3030, Promega, WI).

Results: Animals tolerated cap1-xRNA well without remarkable clinical signs compare to PBS controls. The cap1-xRNA construct remains 10-fold above the PBS control at 240 h. Relevant samples in plot: PBS (background), cap1=cap1-xRNA, identical as in plot above.

As shown by data provided, the replacement of conventional 3′ ends of mRNAs by the synthetic 3′ termini according to the invention does not only provide enhanced and prolonged translation in in vitro systems but also translates to in vivo models (here shown for mammals) clearly indicating the high therapeutic potential of this technology in providing long lasting mRNA-like molecules for the production of e.g., therapeutic peptides.

### Example 6

### Enhancing mRNA Stability Through CDS Modification: a Comprehensive Evaluation Using mRNA Display

In the context of the present invention, we harness the molecular strategy known as mRNA display to generate extensive peptide libraries. This technique relies on the in-vitro translation of an mRNA library into a corresponding peptide library, where each peptide molecule is covalently linked to its encoding mRNA. The covalent linkage is mediated by introducing a puromycin moiety at the 3′ end of each mRNA molecule. During translation, as the ribosome reaches the end of the coding sequence, the puromycin intercalates the ribosome, forming a peptide bond with the last amino acid and establishing a physical connection between the peptide and mRNA.

The disclosed method utilizes mRNA display to assess constructs stability over time. The constructed libraries are transfected into cells and isolated at multiple post-transfection time points, subsequently pooled and purified using Streptavidin beads. Subsequent sequencing enables the determination of sequence abundance at each time point (tx) relative to the t0 library. This information is employed to compute a Stability Score, providing valuable insights into the relative stability of a given mRNA sequence in transfected cells over time.

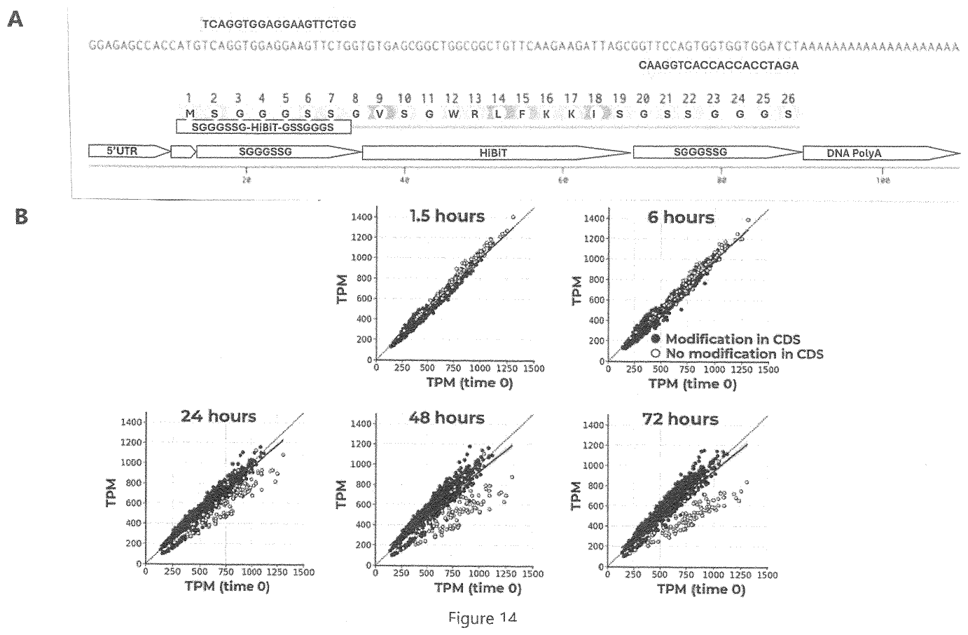

The library composition, as detailed in Table 7, maintains a consistent 5′ UTR for all sequences unless specified otherwise, as illustrated in FIG. 14A.

TABLE 7
Description of the 2000 test library sequences.

| Group name | Number | Description |
|---|---|---|
| HIBIT test | 1,439 | Sequences that are translated to HiBiT, but differ by the |
| | | specific codons that encode the HiBiT peptide. This set |
| | | consists of all the possible sequences generated from fixing |
| | | the codons of first six amino acids (M-V-S-G-W-R), and |
| | | permuting all possible codon options for amino acids at |
| | | positions 7-12 (L-F-K-K-I-S). Alternatively, fixing the second |
| | | half of HiBiT (position 7-12), and permuting all possible |
| | | codon options at positions 2-6 (position 1 remains with the |
| | | start codon ATG). |
| sequences | | The WT HiBiT is encoded as: SEQ ID NO 56 |
| | | ATG-GTG-AGC-GGC-TGG-CGG-CTG-TTC-AAG-AAG-ATT- |
| | | AGC |
| HIBIT frequent | 35 | Sequences that get translated to HiBiT, and encode HiBiT |
| codons | | using codons that are frequent in humans. The codons for |
| | | this group of sequences were permutations of all the codons |
| | | with frequency within the range defined in Table 8. |
| HiBiT rare | 108 | Sequences that get translated to HiBiT, and encode HiBiT |
| codons | | using codons that are rare in humans. The codons for this |
| | | group of sequences were permutations of all the codons |
| | | with frequency within the range defined in Table 8. |
| Premature stop | 108 | Sequences that start getting translated to HiBiT but then |
| codon | | stop because of a premature stop codon (permuting all |
| | | three stop codons at all positions, including the start codon |
| | | position). The other positions have randomly chosen codons |
| | | that encode HiBiT. Since some of the sequences had a stop |
| | | codon in-place of the unsequenced start-codon, a specific |
| | | combination for amino acids S,R,L was chosen to encode |
| | | this group, as defined in Table 4. |
| No start codon | 90 | Sequences that get translated to HiBiT, but instead of the |
| | | regular start codon ATG, have one of: V,I (hydrophobic |
| | | amino acids), K, R (charged amino acids), and A, G (neutral |
| | | amino acids). All of the alternative start codons were |
| | | encoded using the rarest codons as defined by the codon |
| | | usage database. The other positions have randomly chosen |
| | | codons that encode HiBiT. Since the start codon is not |
| | | sequenced, a specific combination for amino acids S,R,L was |
| | | chosen to encode this group, as defined in Table 8. |
| Poly Lys/Pro | 60 | Sequences with a start codon followed by: (1) poly-Lysine |
| | | sequence; (2) poly-Proline sequence, (3) five poly-Lysine |
| | | sequence and six HiBiT WT second half; (4) five poly-Proline |
| | | sequence and six HiBiT WT second half; (5) five HiBiT WT first |
| | | half and six poly-Lysine sequence; and (6) five HiBiT WT first |
| | | half and six poly-Proline sequence. All the Proline and Lysine |
| | | amino acids were randomly encoded using all possible |
| | | codons. |
| 5′ UTR-in-house* | 80 | 16 different 5′ UTRs were chosen based on low expression |
| | | tested at in-house experiments |
| 5′ UTR-Sample* | 50 | 10 different 5′ UTRs were chosen with lowest MRL from |
| | | Sample et al. published data. |
| 5′ UTR-poly nt* | 20 | 4 different 5′ UTRs of poly A/T/G/C. |
| 5′ UTR-loop* | 10 | Two 5′ UTRs that create a stable secondary structure: 1) |
| | | stem-loop, and 2) double helix (sdRNA). This was achieved |
| | | by creating base-complementation with the start codon and |
| | | 5′ linker sequence, or just with the 5′ linker (creating a loop |
| | | of size three). |

*All the sequences with different 5′ UTR had a G at the 5′ end. Their coding region was comprised of randomly selected codons encoding HiBiT. Since the 5′ UTR is not sequenced, a specific combination for amino acids S,R,L was chosen to encode this group, as defined in Table 2.

TABLE 8
Watermark signatures for distinguishing
changes in unsequenced regions

| Group name | Codon combination |
|---|---|
| Premature stop codon | S: AGT, R: CGC, L: TTG |
| No start codon | S: TCA, R: CGA, L: CTC |
| All the groups with different 5′ UTR | S: TCT, R: AGA, L: TTA |

The experimental procedure involved ordering the library from Twist Biosciences, PCR amplifying it, and conducting in-vitro transcription using it as a template. The resultant constructs pool underwent purification, enzymatic capping (Cap-1), and ligation to a DNA oligo containing a 3′-Puromycin. Enzymatic capping employed the One-Step Capping and 2′-O-Methylation protocol and reagents (Vaccinia Capping Enzyme and mRNA Cap 2′-O-Methyltransferase) from NEB. Following isolation and purification through PAGE, the library, comprising 2000 sequences, underwent in-vitro translation in A549 cells. Subsequent incubation under high salt conditions at room temperature, library preparation (RT and PCR amplification), and sequencing occurred at intervals of 1.5 hours, 6 hours, 24 hours, 48 hours, or 72 hours.

Notably, the absence of a premature stop codon results in a coding sequence (CDS) modification due to the presence of DNA nucleotides and Puromycin at the 3′ end of a CDS. Conversely, the presence of a premature stop codon induces a CDS without a chemical modification.

This property was exploited to evaluate the impact of the CDS modification on mRNA stability. For each sequence in each library, the transcripts per million (TPM) count was extracted to assess stability. Specifically, TPM values at time=0 (input library) were compared to TPM values at subsequent time points (time>0). The results, illustrated in FIG. 14B, clearly demonstrate that the presence of a CDS modification significantly enhances mRNA stability, with the effect intensifying over time. Sequences lacking a CDS modification exhibited markedly reduced TPM values after 24, 48, and 72 hours, contrasting sequences with a CDS modification that maintained relatively consistent TPM values throughout the experiment, indicating sustained stability.

1. An artificial polynucleotide molecule comprising or consisting of
i. a polynucleotide M comprising or consisting of at least one open reading frame (ORF), and
ii. a synthetic 3′ terminus comprising or consisting of at least one polynucleotide P, wherein, optionally,
the 3′ end of the polynucleotide M (i) is linked to the synthetic 3′ terminus (ii), and/or
said at least one polynucleotide P has a length of 4 nt and about 1000 nt, and comprises at least one DNA nucleotide, modified RNA, modified DNA or XNA nucleotide, non-natural backbone modification, modified nucleotide, modified backbone linkage, or any combination thereof.

2. The artificial polynucleotide molecule of claim 1, wherein said synthetic 3′ terminus (ii) increases stability and/or translational efficiency and/or bioavailability and/or reduces toxicity and/or immunogenicity of the artificial polynucleotide molecule.

3. The artificial polynucleotide molecule of claim 1, devoid of a canonical poly(A) tail.

4. The artificial polynucleotide molecule of claim 1, wherein said synthetic 3′ terminus (ii) is devoid of non-modified natural RNA nucleotides.

5. The artificial polynucleotide molecule of claim 1, wherein the polynucleotide M (i) is RNA.

6. (canceled)

7. (canceled)

8. (canceled)

9. (canceled)

10. (canceled)

11. (canceled)

12. (canceled)

13. The artificial polynucleotide molecule of claim 1, wherein the ORF is devoid of a STOP codon.

14. (canceled)

15. (canceled)

16. (canceled)

17. The artificial polynucleotide molecule of claim claim 1, wherein the ORF at the last codon comprises at least one modified nucleotide, or at least one DNA nucleotide.

18. (canceled)

19. (canceled)

20. The artificial polynucleotide molecule of claim 17, wherein the last codon of the ORF consists of three adenine deoxynucleotides.

21. (canceled)

22. The artificial polynucleotide molecule of claim 1, wherein the at least one ORF encodes at least one therapeutic polypeptide.

23. (canceled)

24. The artificial polynucleotide molecule of claim 1, wherein the at least one polynucleotide P comprises or consists of at least 50%, preferably at least 60%, preferably at least 70%, preferably at least 80%, preferably at least 90%, preferably at least 95%, or most preferably 100% modified and/or unmodified DNA nucleotides.

25. The artificial polynucleotide molecule of claim 24, wherein said modified DNA nucleotides are selected from the group of DNA nucleotides with modifications to the ribose, modifications to the phosphate backbone, modifications to the nucleobases, stereoisomerization of the entire nucleotide, or any combination thereof.

26. (canceled)

27. The artificial polynucleotide of claim 25, wherein the modification to the ribose is 2′-H(DNA).

28. (canceled)

29. The artificial polynucleotide of claim 25, wherein the modification to the phosphate backbone is phosphonothioate links.

30. (canceled)

31. The artificial polynucleotide molecule of claim 30, wherein the modification to the nucleobase is Biotin addition.

32. (canceled)

33. (canceled)

34. (canceled)

35. (canceled)

36. (canceled)

37. (canceled)

38. (canceled)

39. (canceled)

40. (canceled)

41. The artificial polynucleotide molecule of claim 1, wherein said synthetic 3′ terminus (ji) further comprises at least one building block selected from the group of L and C or any combination thereof, wherein L denotes a chemical linker and C denotes a one or more monomers.

42. (canceled)

43. (canceled)

44. The artificial polynucleotide molecule of claim 41, wherein the synthetic 3′ terminus (ii) further comprises at least one building block L selected from the group of L and C or any combination thereof, wherein L denotes a chemical linker and C denotes one or more monomers.

45. The artificial polynucleotide molecule of claim 44, wherein the at least one building block L denotes a chemical spacer Sp9.

46. (canceled)

47. (canceled)

48. (canceled)

49. (canceled)

50. (canceled)

51. (canceled)

52. (canceled)

53. The artificial polynucleotide molecule of claim 44 wherein the at least one building block C is ACC-Puromycin.

54. (canceled)

55. (canceled)

56. (canceled)

57. (canceled)

58. (canceled)

59. (canceled)

60. A therapeutic polynucleotide construct comprising the artificial polynucleotide molecule of claim 1, wherein the at least one ORF encodes at least one therapeutic polypeptide.

61. A pharmaceutical composition comprising the therapeutic polynucleotide construct of claim 60.

62. (canceled)

63. (canceled)

64. (canceled)

65. (canceled)

66. (canceled)

67. (canceled)

68. (canceled)

69. (canceled)
